(19) United States
(12) Patent Application Publication
(10) Pub. No.: US 2006/0141510 A1
(43) Pub. Date: Jun. 29, 2006

(54) Method for information processing with nucleic acid molecules

(75) Inventors: Akira Suyama, Hachioji-shi (JP); Nao Nitta, Tokyo (JP)

Correspondence Address: Scully, Scott, Murphy & Presser, 400 Garden City Plaza, Garden City, NY, 11530-3319, US

(73) Assignee: Olympus Corporation, Tokyo (JP)

(21) Appl. No.: 11/290,259

(22) Filed: Nov. 30, 2005

Related U.S. Application Data
(63) Continuation of application No. PCT/JP04/00952, filed on Jan. 30, 2004.

(30) Foreign Application Priority Data
May 30, 2003 (JP) 2003-155988

(51) Int. Cl. C12Q 1/68; G06F 19/00

(52) U.S. Cl. 435/6; 702/20

(57) ABSTRACT

The present invention is directed to provide an information processing method using autonomously workable nucleic acids, and a molecular computer to carry out operations with the method. Objectives above mentioned may be achieved with following method. The present invention provides an information processing method carrying out operations with functions receiving an argument and returning a return value through chemical reactions of molecules, comprising (a) inputting a first encoded nucleic acid defined in correspondence to a first degradable single stranded nucleic acid as an argument (b) carrying out an operation with functions defined in correspondence to chemical reactions of operator nucleic acids based on the argument (c) obtaining a second encoded nucleic acid defined in correspondence to a second single stranded nucleic acid as a return value. Furthermore, the invention provides a molecular computer designed on the basis of the method.

### CROSS REFERENCE TO RELATED APPLICATIONS

This is a Continuation Application of PCT Application No. PCT/JP2004/000952, filed Jan. 30, 2004, which was published under PCT Article 21(2) in Japanese.

This application is based upon and claims the benefit of priority from prior Japanese Patent Application No. 2003-155988, filed May 30, 2003, the entire contents of which are incorporated herein by reference.

### BACKGROUND OF THE INVENTION

1. Field of the Invention

The present invention relates to a DNA computer.

2. Description of the Related Art

A DNA computer is known as a unique attempt to utilize the characteristics of biomolecules. Calculation in DNA computers involves artificial incorporation of input values and programs into DNA sequence and appropriately combining the resulting DNA with various reactions such as enzyme reactions (ex. DNA modification enzymes and restriction enzymes) and hybridization reactions with other DNAs.

The history of DNA computers dates to demonstrating by Adleman that the experimental system with DNAs can be used to solve a mathematical problem (Adleman LM, Molecular computation of solutions to combinatorial problems., “Science”, (USA), 1994; 266(5187), p. 1021-4). In this study, he solved a mathematical problem, directed Hamiltonian Path Problem, using an experimental system with DNA molecules. In addition, in the year after, Lipton reported the solution for satisfiability problem with a DNA computer (Lipton R J, DNA solution of hard computational problems., “Science”, USA, 1995; 268(5210), p. 542-5). Many kind of Computational algorithms for a DNA computer have been proposed, which include the technique based on an elongation reaction in single DNA molecule (Sakamoto K, Gouzu H, Komiya K, Kiga D, Yokoyama S, Yokomori T, Hagiya M,, Molecular computation by DNA hairpin formation., “Science”, USA, 2000; 288 (5469), p. 1223-6, akamoto K, Kiga D, Komiya K, Gouzu H, Yokoyama S, Ikeda S, Sugiyama H, Hagiya M, State transitions by molecules., “Biosystems”, 1999; 52 (1-3), p. 81-91) and the approach with hairpin structure in single stranded DNA (Sakamoto K, Kiga D, Komiya K, Gouzu H, Yokoyama S, Ikeda S, Sugiyama H, Hagiya M, State transitions by molecules. “Biosystems”, 1999; 52(1-3), p. 81-91), the technique to identify the appropriate solution on the solid phase using DNA as memories (Liu Q, Wang L, Frutos A G, Condon A E, Corn R M, Smith L M DNA computing on surfaces., “Nature”, UK, 2000; 403(6766), p. 175-9, ang L, Hall J G, Lu M, Liu Q, Smith L M A DNA computing readout operation based on structure-specific cleavage., “Nat Biotechnol”, UK, 2001; 19(11), p. 1053-9) and the method involving insertion of double stranded DNA into plasmids and cleavage of double stranded DNA. Furthermore, DNA computation is expanding its scope into further area including some reports, such as RNA based, instead of DNA, molecular computation (Faulhammer D, Cukras A R, Lipton R J, Landweber L F Molecular computation: RNA solutions to chess problems., “Proc Natl Acad Sci“, USA, 2000; 97(4), p. 1385-9), the technique based on nanostructure formed with self-assembly of DNA (Mao C, LaBean T H, Relf J H, Seeman N C, Logical computation using algorithmic self-assembly of DNA triple-crossover molecules., “Nature”, UK, 2000; 407(6803), p. 493-6).

In almost conventional DNA computation including Adleman's studies, DNA molecules having specific sequence are used as input data, and programs are defined with protocols of subsequent biochemical operation steps. Recently, some scientists are studying for achieving large scale calculation with robotic technologies for automatization of various reactions (Japanese patent publication (Tokkai) 2002-318992, (Tokkai) 2002-181813, Morimoto N, Kiyohara H, Sugimura N, Karaki S, Nakajima T, Makino T, Nishida N, Suyama A, Automated processing system for gene expression profiling based on DNA computing technologies., “Eighth International Meeting on DNA Based Computers”, Japan, 2002; Hokkaido University, Suyama A, Programmable DNA computer with application to mathematical and biological problems., “Eighth International Meeting on DNA Based Computers”, Japan, 2002; Hokkaido University). From a different viewpoint, some scientists are also working on studies for an autonomously working molecular computer. This type of computers, which can execute programs without the need of extraneous handling for a reaction solution to initiate reactions, work autonomously and output calculation results under certain conditions by addition of input data and calculation programs as DNA molecules into a reaction solution, and one of such computer technologies, developed using turing machines as a model, has been published (Benenson Y, Paz-Elizur T, Adar R, Keinan E, Livneh Z, Shapiro E, Programmable and autonomous computing machine made of biomolecules. “Nature”.UK. 2001; 414(6862), p. 430-4). An autonomously running molecular computer is attracting the attention because of its potential to calculate in an environment where conventional computers could never work, such as interior of living cells.

The main purpose of such studies for DNA computers is to achieve large scale parallel computation. This is based on the idea that in a test tube, in which a large number of DNA molecules can co-exist, and chemical reactions corresponding to calculation processes are carried out concurrently with assembly of the DNA molecules into each of which an initial values for calculation or a computation program itself is applied, which enables to carry out computation with very wide-ranging initial values or computation programs all at once in parallel. As described above, the studies have been made to develop the system to execute mathematical calculations such as parallel computation using parallelable reactions characterizing the DNA computing system.

While the studies for application of bioreactions to mathematical purposes have been attracted a lot of attention due to their unique ideas and potential, studies for practical applied technologies have not progressed and their capability are still unclear at the present stage. On the other hand, conventional computers, in particular, using electronic signals are improved in their processing capacity year by year, suggesting the low potency of the molecular computers to exceed the conventional ones in their processing capacity and correctness. There is a need of finding the suitable field for the molecular computers, different from the conventional computer-applied fields, to provide their best effect. In the meantime, some scientists is starting the studies to apply DNA computers to gene expression analysis and SNPs analysis (Nishida N, Wakui M, Tokunaga K, Suyama A, Highly specific and quantitative gene expression profiling based on DNA computing., “Genome Informatics”, 2001; (12), p. 259-260, Mills A P Jr, Gene expression profiling diagnosis through DNA molecular computation., “Trends Biotechnol”, 2002; 20(4), p. 137-40). These may be promising as the applicable fields suitable for unique property of molecular computers in which biomolecules can be used as input data directly. However, conventionally molecular computers has bee proposed which cannot work autonomously as applicable ones to bioanalysis, thus their application is restricted.

Accessing to information comprised in a nucleic acid involves hybridization reactions between nucleic acids, which cause formation of a stable hybrid between nucleic acids at the site, blocking further accessing to information without any treatments. However, it is desirable to construct nucleic acids-information utilizing molecular computers in which the information can be accessed repeatedly like chain reaction. To solve the problem, some processes are needed to return the inaccessible information in double stranded nucleic acid molecules to be in accessible state again. In conventional DNA computers, this process often involves denaturing of nucleic acids with heating. However, this procedure is incompatible with an autonomously running molecular computer because extraneous temperature control is needed. The key factor to realize an autonomously running molecular computer is to return information enclosed in double stranded nucleic acid to an available state again by using molecular reactions, for example enzyme reactions. One example of a molecular computer is achieved by Shapiro et al., who has succeeded to realize an autonomous running molecular computer by digesting double stranded DNA with restriction enzymes to expose single stranded DNA at the digested site (Y. Benenson et al, DNA molecule provides a computing machine with both data and fuel, “Proc. Natl. Acad. Sci.”, 2003; 100, p. 2191-6).

### BRIEF SUMMARY OF THE INVENTION

In consideration of the situation above, the present invention is directed to provide an information processing method using autonomously workable nucleic acids, and a molecular computer to carry out operations with the method.

In view of the situation above, the present invention is directed to provide an information processing method using autonomously workable nucleic acids, and a molecular computer to carry out operations with the method.

Procedures to Solve the Problems

The assignments above can be achieved by procedures, for example, below. The present invention provides an information processing method carrying out operations with functions receiving an argument and returning a return value through chemical reactions of molecules, comprising:

(a) inputting a first encoded nucleic acid defined in correspondence to a first degradable single stranded nucleic acid as an argument:

(b) carrying out an operation with functions defined in correspondence to chemical reactions of operator nucleic acids based on the argument:

(c) obtaining a second encoded nucleic acid defined in correspondence to a second single stranded nucleic acid as a return value.

### BRIEF DESCRIPTION OF THE SEVERAL VIEWS OF THE DRAWING

FIG. 1 shows a diagram of retrovirus genome replication.

FIG. 2 shows a processing flow of basic processing in a method of the invention.

FIG. 3 shows a processing flow of basic processing in a method of the invention.

FIGS. 4A to 4C show diagrams of reactions used for a molecular computer.

FIGS. 5A and 5B show conceptual diagrams of an information processing method of the invention.

FIGS. 6A to 6E show diagrams of various types of basic functions.

FIGS. 7A to 7C show diagrams of a gene analysis procedure with gene encodings and logic operation.

FIGS. 8A to 8C show summaries of a gene analysis procedure with a neural network.

FIGS. 9A and 9B show operator nucleic acids for detection with FRET.

FIG. 10 shows a result of measurement of RNA dependent DNA polymerase activity under the high-temperature reaction condition.

FIG. 11 shows a result of measurement of DNA dependent RNA polymerase activity under the high-temperature reaction condition.

FIG. 12 shows results of measurement of DNA dependent DNA polymerase activity under the high-temperature reaction condition.

FIG. 13 shows a schematic view of TGTP-P1 primer, which was used in a method of the invention.

FIG. 14 is a photo of electrophoresis showing activity and specificity of elongation with TGTP-P1 primer.

FIG. 15 shows a schematic view of a gene encoding function to detect the TGTP gene expression.

FIG. 16 shows an output result from an operation with a function for detection of TGTP gene expression.

FIG. 17 shows an output result from an operation with a function for detection of TGTP gene expression.

FIG. 18 shows a schematic view of an encoded nucleic acid used for reverse transcription reaction of a path containing multiple RNA molecules.

FIG. 19 shows a photo of electrophoresis of reaction products of reverse transcription reaction of a path containing multiple RNA molecules.

FIG. 20 shows a schematic view of an operator nucleic acid for a logic operation reaction.

FIG. 21 shows a result of a logic operation reaction.

FIG. 22 shows an operator nucleic acid used for Amplify function to amplify sense strand TGTP RNA.

FIG. 23 shows a photo of electrophoresis demonstrating a result of an operation with Amplify function to amplify sense strand TGTP RNA.

FIG. 24 shows an example of the case using multilayered functions.

FIG. 25 shows a result of detection for Code[4, 5, 6]RNA in reaction products.

FIG. 26 shows a result of detection for Code[3, 2]RNA in reaction products.

### DETAILED DESCRIPTION OF THE INVENTION

The inventors made studies of solutions to this problem and, as a result, accomplished the present invention based on the following idea.

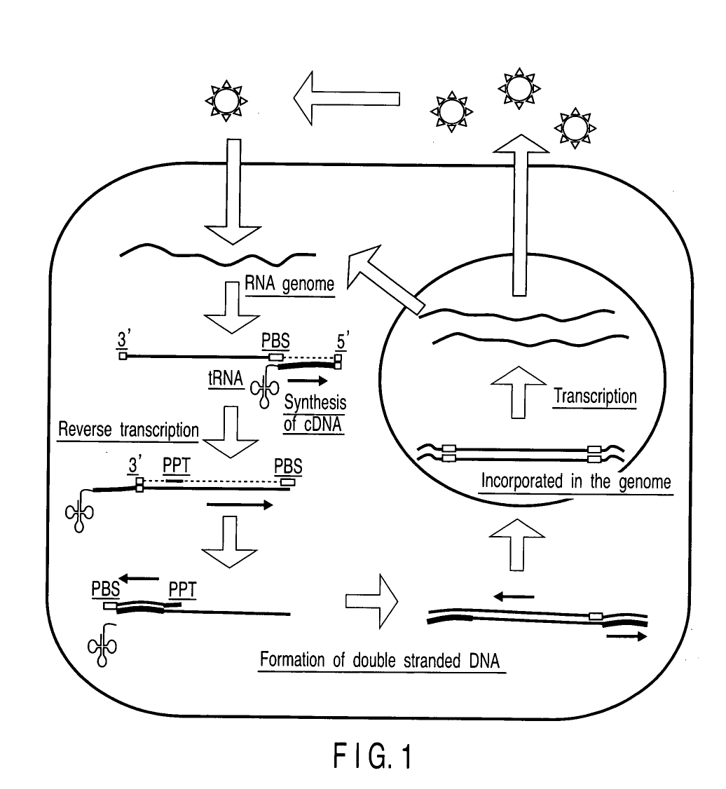

It is known that retrovirus, one of RNA genome-containing virus, replicates within host cells (FIG. 1). Replication of RNA genome is led by reverse transcription of RNA into CDNA with RNA dependent DNA polymerase activity of reverse transcriptase. At first tRNA hybridizes to primer binding site (PBS) in genome to act as a primer. At this site, reverse transcription is initiated, which provides cDNA synthesis leading to 3′-end of the genome, followed by strand-transfer into 5′-end and subsequent further reverses transcription. As a result, the first strand cDNA is formed in full length genome (Mak et al. Primer tRNAs for reverse transcription. J Virol November 1997; 71(11):8087-95). RNA strand in the formed DNA-RNA hybrid is removed with RNaseH activity of reverse transcriptase. Then hybridization of the remaining single stranded DNA occurred with DNA dependent DNA polymerase activity, and incorporation of the resulting double stranded DNA into genome leads to initiation of transcription of the genome sequence at promoter region. As a result, genomic RNA is generated which have identical sequence to original genome. In addition, long terminal repeat (LTR) retrotransposon existing within a cell is known to replicate in the similar mechanism, which involves transcription of a sequence in double stranded DNA into single stranded RNA, followed by further reverse transcription and formation of double stranded DNA (Wilhelm Reverse transcription of retroviruses and LTR retrotransposons. Cell Mol Life Sci August 2001; 58(9):1246-62).

Retrovirus genome replication above comprises 4 characteristic reactions. The first reaction is a reverse transcription reaction by RNA dependent DNA polymerase activity. The second is formation of double stranded DNA by DNA dependent DNA polymerase activity. The third is a transcription reaction by DNA dependent RNA polymerase activity. Furthermore, in replication of full-length genome, RNaseH activity is also important to remove RNA strand in DNA-RNA hybrid during reverse transcription and formation of double stranded DNA. Genome amplification is achieved by combination of these 4 reactions. Looking such a series of systems as a kind of computer, retrovirus may be regarded to execute the program receiving its own genome RNA as “an input” and returning replicated RNA having an identical sequence to the input with above 4 reaction activity in a host cell, “hardware”.

Appropriate combination of above 4 reactions may also enable to allow such systems to execute a program different from self-genome-replicating program of retrovirus. Therefore, the invention has attempted to design a molecular computer comprising such 4 reactions. The molecular computer designed herein uses a reaction solution, as hardware, in which RNA dependent DNA polymerase, DNA dependent DNA polymerase, DNA dependent RNA polymerase and RNaseH activities are made active concurrently. To this hardware, RNA samples, as an input data, are provided to carry out operations with “functions” using RNA molecules as arguments and return values. In this invention, some examples are defined as underlying functions working in this hardware. Furthermore, combining these functions accordingly enables to construct programs, which are also applicable to gene expression analysis and like. Such molecular computers may exert different effects depending on introduced programs. Therefore, it may be a programmable general-purpose molecular computer.

Among reverse transcription activity, double-stranded-DNA formation activity, transcription activity and RNaseH activity, all of which are comprised in retrovirus genome amplification system, transcription activity and RNaseH activity may be listed as the most characteristic reactions in application of this mechanism to an autonomous running molecular computer. In retroviral typed molecular computers, key factors to allow molecular computers to run autonomously are transcription activity to separate single stranded RNA from double stranded DNA molecules and RNaseH activity to remove only RNA strand from DNA-RNA hybrid to leave single stranded DNA.

Based on ideas above, the present inventions has developed an information processing method for carrying out operations with functions receiving arguments and returning return values based on realization of autonomous reactions, which involves molecular chemical reactions with enzymes having polymerase activities such as DNA dependent DNA polymerase, RNA dependent DNA polymerase and DNA dependent RNA polymerase activity, RNaseH activity and like respectively.

As used herein, an “autonomous” reaction refers to that a reaction product can be obtained without extraneous handlings such as separation and isolation of nucleic acids in the course of molecular chemical reactions. In turn, an operation with a function outputting a return value against an input argument can be carried out without extraneous handlings.

As used herein, a “nucleic acid” includes all kind of DNA and RNA, including cDNA, genomic DNA, synthetic DNA, mRNA, total RNA, hnRNA and synthetic RNA, as well as artificial nucleic acids, such as peptide nucleic acids, morpholino nucleic acids, methylphosphonate nucleic acids and S-oligo nucleic acids. In the specification, “nucleic acid”, “nucleic acid molecule” and “molecule” are used synonymously each other.

As used herein, the both terms of “base sequence” and “sequence” refer to the array of bases composing specific nucleic acid.

Hereinafter, preferred embodiments of the present invention will be described with referent to the drawings.

According to preferred embodiments of the invention, an information processing method using nucleic acids is provided.

The invention discloses an autonomously-executable method for data processing and gene analysis involving carrying out calculation with nucleic acids. Also, an autonomous process of reactions is achieved by describing data and programs with nucleic acid molecules and replacing operations defined in the program with molecular reactions.

At first, as development of an information processing method of the invention, detail of operations performed is converted into executable data format for molecular chemical reactions. Specifically, before execution of an operation with molecular reactions, information is converted into encoded nucleic acids which are generated by pre-association of a molecule and a specific code. Data, such as parameters and constants for operations, are replaced to encoded nucleic acids according to conversion rules. Then, arithmetic processing with these encoded nucleic acids is conducted to obtain outputs with the encoded nucleic acids. The operations are accomplished by conversion of the resulting encoded nucleic acids into information pre-associated to them.

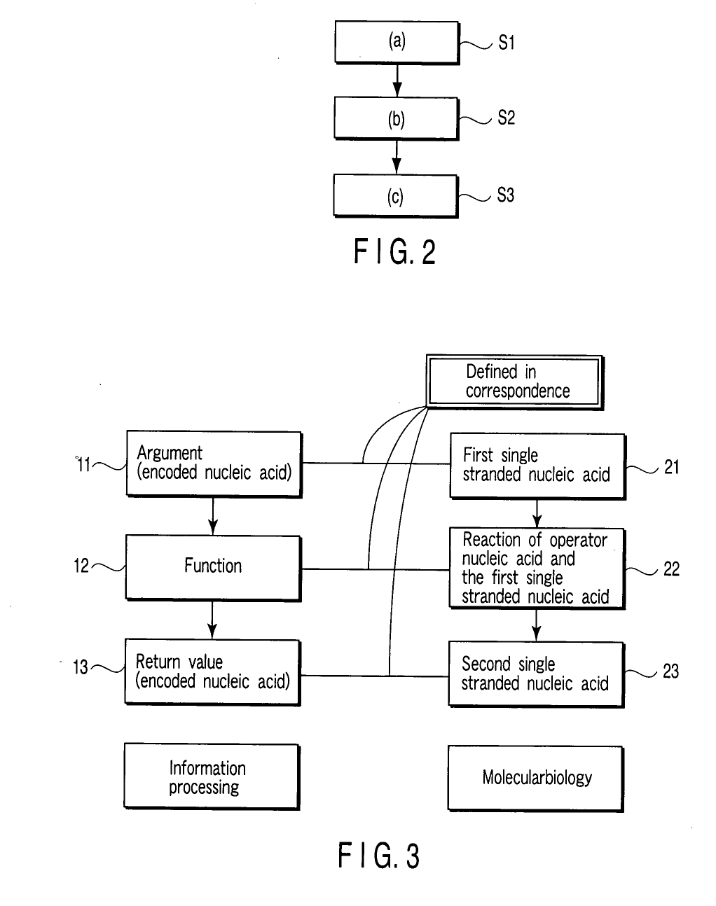

The first embodiment of the invention will be described according to processing flows in FIGS. 2 and 3.

FIGS. 2 and 3 show steps of information processing involving an operation with functions receiving arguments and returning return values.

(S1) is a step for inputting argument 11. Specifically, an encoded nucleic acid defined in correspondence to degradable single stranded nucleic acid 21 as an argument.

(S2) is a step for carrying out an operation with function 12. Specifically, an operation is carried out, based on argument 21, using function 12 defined in correspondence to chemical reaction 22 with operator nucleic acid 22. “Operator nucleic acids” are various nucleic acids designed to react with input single stranded nucleic acid 21 etc to produce specific reaction products through given reactions. In turn, they are nucleic acids having sequence required to initiate chemical reactions corresponding to functions, and, for example, they act as primers and promoters. Plural operator nucleic acids may be available, which may be used to carry out single function.

(S3) is a step for obtaining return value 13 of the function. Specifically, encoded nucleic acid 13 defined in correspondence to single stranded nucleic acid 23 is obtained in the step.

Herein, “defined in correspondence to” describes correspondence of a manipulation in information processing to a manipulation in a chemical reaction of nucleic acids. It means that encoded nucleic acid (argument) 11, an operation with function 12 and return value 13 in information processing correspond to the degradable single nucleic acid 22, used in a chemical reaction, chemical reaction 22 with operator nucleic acids in a chemical reaction and degradable single stranded nucleic acids etc., and single stranded nucleic acid 23, which is a reaction product in a chemical reaction, respectively.

In step (S1), an input argument is not required to be an encoded nucleic acid defined in correspondence to a degradable single stranded nucleic acid, thus a degradable single nucleic acid itself can be input directly as an argument. In this case, arithmetic processing is carried out with a degradable single stranded nucleic acid itself to obtain an output with encoded nucleic acids. In addition, not only encoded nucleic acid 13 defined in correspondence to the second single stranded nucleic acid 23 but also the second single stranded nucleic acid may be obtained directly as a return value of a function obtained in (S3). However, when an operation with functions is carried out as information processing method, either an argument or a return value should be an encoded nucleic acid in which a molecule is pre-associated with a specific code.

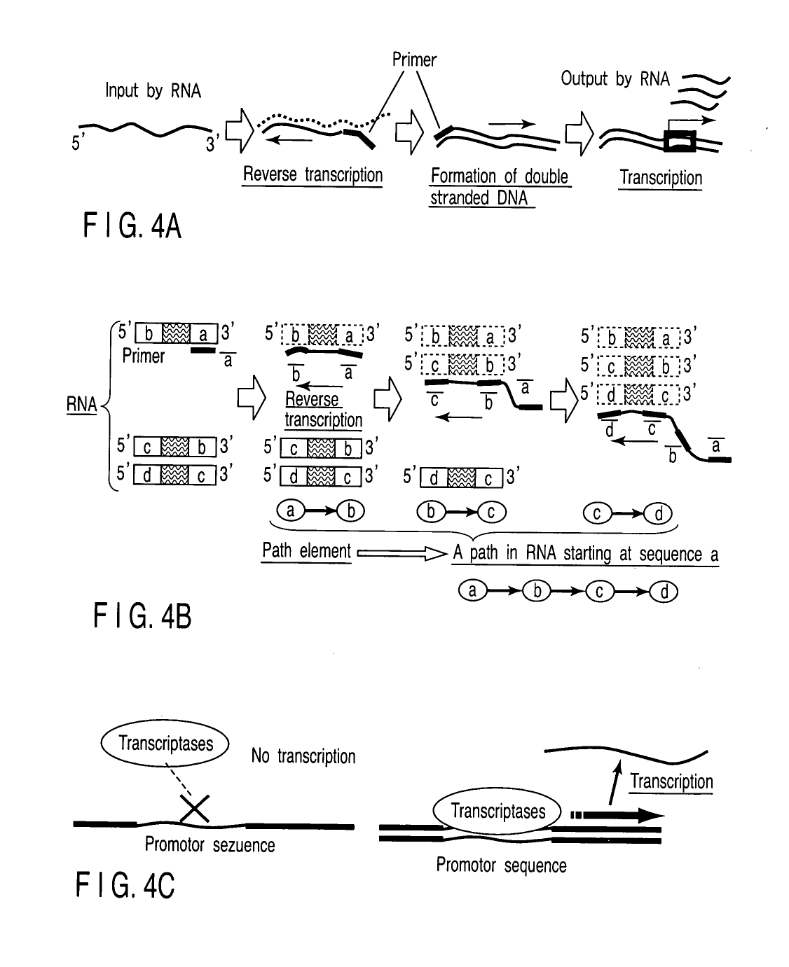

An example of chemical reactions used in the invention is showed in FIG. 4A.

A method of the invention provides an “input” as a degradable single stranded nucleic acid (ex. a RNA molecule). “Input of an argument” in information processing corresponds to adding a degradable single stranded nucleic acid to a reaction solution. Hereinafter, a method of the invention will be described taking the case of RNA used as a degradable single stranded nucleic acid, as an example.

The presence of an operator nucleic acid corresponding to an input RNA molecule (the primer showed in FIG. 4A) leads to reverse transcription resulting in reading of the input. At the same time, RNA strand of a DNA-RNA hybrid generated during reverse transcription would be degraded with RNaseH activity. The degrading with RNaseH corresponds to erasing of input information in conventional information processing.

In conventional DNA molecules-based information processing methods, input DNAs was not digested and it was still left in a reaction system after read of input DNAs. Thus, when the DNAs were undesired in subsequent reactions, complicated handlings were required to remove the DNAs. Such separating treatments are accompanied by a series of extraneous handlings, making it difficult to realize autonomous running. For example, robotic separating manipulations were required to automatize separating treatment. In contrast, the use of degradable nucleic acids, for example RNA molecules in the case of which only input RNA can be easily removed with RNaseH activity, would allow autonomous initiation of reactions in a reaction system. Despite RNA molecules used as degradable single stranded nucleic acids in the invention, other degradable nucleic acids may also be used.

Herein, “degradable” refers to that only “degradable single stranded nucleic acids” are degraded while other nucleic acids are not degraded. It means that, in particular, under the condition that operation nucleic acids are not degraded, only “degradable single stranded nucleic acids” are degraded selectively. For example, when DNA is used as an “operator nucleic acid”, RNA would be “degradable” because RNA would be selectively degraded with RNaseH. Furthermore, when RNA is used as an “operator nucleic acid” with addition of a pure deoxyribonuclease, DNA can be degraded selectively, thus DNA would be a “degradable” nucleic acid in such a condition. Therefore, “degradable” may have a relative concept.

Other examples of “degradable” nucleic acids include, but are not limited to, uracil containing DNA used when an operator molecule is DNA (A RACHITT for our toolbox, Nature Biotechnology, April 2001 Volume 19 Number 4 pp 314-315, DNA shuffling method for generating highly recombined genes and evolved enzymes, Nature Biotechnology, April 2001 Volume 19 Number 4 pp 354-359), and DNA and RNA when an operator molecule is Peptide Nucleic Acid.

In a method of the invention, a single stranded nucleic acid is input as an argument. An information processing method with nucleic acids involves hybridization reactions to access the information in nucleic acid sequence. Thus, in the case of the conventional technique using double stranded DNA as an input, reactions to return double stranded DNA into single stranded DNA is required to allow the double stranded DNA to hybridize with an operation nucleic acid. However, in such reactions, a series of extraneous handlings is required to control reaction temperature. Therefore, such reactions, as the separation treatments above, made it difficult to allow a series of reactions to run autonomously.

When nucleic acids are not degradable, accompanied by remaining of hybridized double stranded nucleic acids, the temperature control is required to unwind them into single strand.

In contrast, degradable nucleic acids are used in a method of the invention, resulting in the nucleic acids degraded after hybridization. Thus, the autonomous operations are achieved. In other words, the method enables to leads chemical reactions of operator nucleic acids even at constant temperature, providing autonomously occurring degrading reaction. For example, as discussing in the following examples, autonomous reactions may be achieved at constant temperature, 50° C.

On the other hand, information input as RNA is removed by degrading with RNaseH, and, at the same time, reverse transcripted into a more stable nucleic acid (ex. DNA molecules), allowing them to be stored and saved more stably. In addition, remaining single stranded DNA acts as a primer for yet another RNA, and, thus, may serves repeatedly as an operator nucleic acid. Thus, it would be possible to induce further elongation reaction for the DNA (FIG. 4B). Herein the sequence generated by reverse transcription with the primer (sequence a) along with one or more RNA strands, as described in FIG. 4B, is designated as “a path in RNA starting at sequence a”. The resulting single stranded DNA hybridizes in the presence of a primer complementary to the single stranded DNA (which is also one of operator nucleic acids) to be double stranded DNA. RNA molecules transcripted from this double stranded DNA may be obtained as an output of an operation using functions (FIG. 4A).

It is known that a promoter region has to be double stranded DNA to induce the transcription activity of transcriptases such as T7 RNA polymerase (Milligan et al. Oligoribonucleotide synthesis using T7 RNA polymerase and synthetic DNA templates. Nucleic Acids Res November 1987 11;15(21):8783-98). In the present invention, outputs are controlled based on this characteristic (FIG. 4C). For example, if a promoter sequence incorporated into the primer used as an operator nucleic acid, transcription may not be initiated at the promoter sequence when nucleic acids are single stranded DNA, while they may act as a transcription start site when they become double stranded DNA which is recognized by an enzyme. Such a mechanism may be utilized for the control of output.

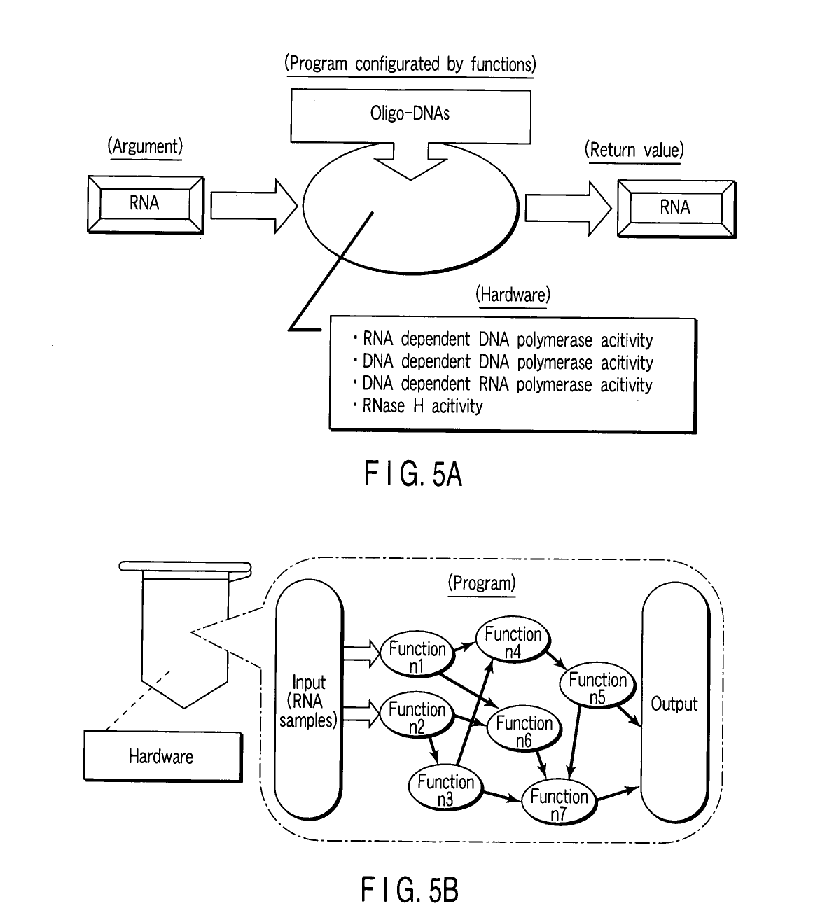

In an information processing method of the invention, whole a series of systems makes one component which receiving inputs with RNA and returning outputs resulted from occurrence of various reactions. As mentioned above, such a component is designated as a “function” receiving an “argument” and returning a “return value” (FIG. 5A).

Obtaining a return value as a single stranded nucleic acid, in an information processing method of the invention, it is easy to access this return value again. In addition, arguments and return values in each function are same kind of molecules (both are RNA, degradable nucleic acids), which allows a return value of one function to be an argument for another one. Thus, a return value from one function may be used as an argument of further function to obtain a further return value. In addition, plural arguments can be used, without limiting to single argument per function. In this case, functions are also defined to use the return values obtained from the plural functions as arguments to obtain further return values. Combining such functions, it may be possible to obtain certain return values. In turn, operations with plural functions can be also carried out following a program described with combination of functions, arguments and return values to extract calculation results as return values.

Assuming that whole a series of systems above is a molecular computer, a reaction solution composed of operator nucleic acids for carrying out operations with desired functions, suitable reaction solution and suitable enzymes would correspond to “hardware” in a computer to execute operations with these functions. A “program” would be defined with operator nucleic acids such as DNA (or RNA) primers and like, determining which reactions will occur (FIG. 5B). The use of an information processing method of the invention provides a molecular computer having the ability to carry out the reactions depending on input of RNA in a reaction solution working as hardware and output the results with RNA (FIG. 5B).

Design of Various Underlying Functions

Specific examples of operations with functions above are given as follows.

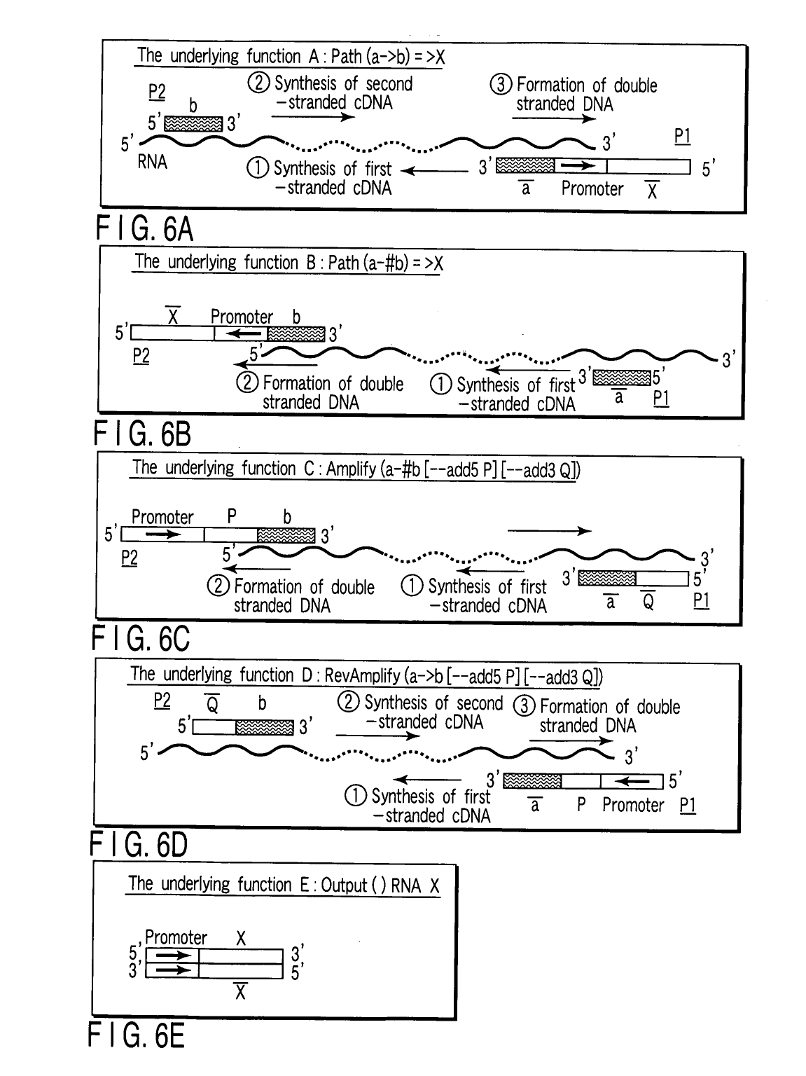

Functions carried out in an information processing method of the invention are defined with operator nucleic acids. Preferably, operator nucleic acids are primers having one or more sequences selected from, for example, sequences acting as a primer for a single stranded nucleic acid, promoter sequences and sequences acting as a primer for any nucleic acid. When arguments are RNA molecules as degradable nucleic acids, two kind of operation nucleic acids, the first primer (P1), which hybridizes with this single stranded RNA and initiate the elongation reaction of DNA to form the first strand cDNA, and the second primer (P2), which hybridizes with the first strand cDNA, are required to carry out an operation with functions according to the invention. When a promoter sequence is incorporated at any site of these primers, hybridization of the primers induces transcription activity. As a result, specific RNAs are output. As the examples of above functions, the following 4 types of functions are considered depending on location and direction of an incorporated promoter sequence (FIGS. 6A, B, C and D). A function receiving no argument is also provided (FIG. 6E). In addition, it may be possible for those skilled in the art to define various functions based on above functions.

Hereinafter, above 5 functions will be described in detail.

The underlying function A: Path (a→b)=>X

The function returns RNA of specified sequence X in the presence of a path in RNA starting at sequence a through sequence b.

P1 is a primer having a promoter sequence in 5′-end direction, a reverse complementary sequence of X at downstream of the promoter sequence and a complementary strand sequence of a at its 3′-end, and primer P2 has the base sequence b (FIG. 6A). The presence of RNA molecules having sequence a in the reaction solution containing P1 and P2, designed like above, induces reverse transcription starting at P1. This reaction may be a reaction proceeding along with multiple RNA molecules as showed in FIG. 4B. Specifically, it includes the case that 3′-end of single stranded cDNA, generated in reverse transcription reaction starting at a primer, binds to another RNA and acts as a primer, resulting in initiation of another reverse transcription. In this case, particularly, the base sequence along with which reverse transcription reaction proceeds (in the case of FIGS. 4B, a→b→c→d) is designated as “a path in RNA starting at sequence a”. In addition, a sequences of RNA molecule consisting of a path (in this case, a→b and c→d) are designated as “a path element”.

Then, the presence of a complementary sequence to sequence B in the single stranded DNA generated by reverse transcription from P1, to which primer P2 binds, induces initiation of synthesis of a second strand DNA. This reaction makes a promoter sequence in P1 double stranded and induces transcription, which provides output of RNA molecules of sequence X located in downstream of the promoter. Therefore, the reaction is a function returning RNA of sequence X when sequence b exists along the path in RNA starting at sequence a.

Underlying function B: Path (a−# b [; b′; b″ . . . ])=>X

This function returns RNA of specified sequence X in the presence of a path in RNA starting at sequence a and ending at sequence b. The terminating condition may be extended in a paratactic manner as “b or b′, b″ . . . ”.

P1 is a primer having complementary strand sequence to a, and P2 is a primer having a promoter sequence in 5′-end direction, sequence X at downstream of the promoter sequence and sequence b at 3′-end of X (FIG. 6B). Here, RNA molecules having sequence an input, reverse transcription would proceeds along with a path in RNA starting at that site. When the path ends at a complementary strand of sequence b, the terminal sequence would bind to sequence B in P2 to act as a primer, resulting in sequence X, located in downstream of the double stranded promoter sequence, transcripted. In P2, the multiple sequences bound with a primer may be aligned as “b, b′, b″ . . . ”. In this case, the terminating condition of the path would be extended in a paratactic manner as “b or b′, b″ . . . ”. Furthermore, P2 itself is not required to be elongated in this function. Thus, special modifications and base sequences may also be added at 3′-end of P2.

Underlying function C: Amplify (a−# b [--add5 P] [--add3 Q])

When there is a path in RNA starting at sequence A and ending at B, this function amplifies RNA of that sequence. In addition, it can also amplify RNA with addition of optional sequence P or Q at 3′- or 5′-end of the amplified sequence.

P1 is a primer having complementary strand sequence to a, and P2 consists of a promoter sequence in 3′-end direction and sequence b at its 3′-end (FIG. 6D). Inputting of RNA molecules having complementary sequence to sequence a here leads to reverse transcription along with a path in RNA. When the path ends at a complementary strand of sequence b, the terminal sequence binds to sequence b in P2 to act as a primer, resulting in RNA having the sequence of complementary strand of the path from a to b (this strand is identical to original input RNA), which located in downstream of double stranded promoter sequence, transcripted. A return value of this function becomes an argument for the same function recursively, and as a result, a loop is formed, which leads to amplification of gene sequence. In addition, a complementary strand sequence to sequence P or Q incorporated into each primer, P1 and P2, as needed, an optional sequence P or Q may be added to 5′-or 3′-end of an output RNA molecule.

Underlying function D: RevAmplify (a→b [--add5 P] [--add3 Q])

When there is a path in RNA starting at sequence A through sequence b, this function amplifies RNA of its reverse complementary strand sequence. In addition, an optional sequence, P or Q, may be added to 3′- or 5′-end of the amplified sequence.

P1 consists of a promoter sequence in 3′-end direction and a complementary strand to sequence a at its 3′-end, and P2 has sequence b (FIG. 6D). Inputting of RNA having sequence a here leads to reverse transcription proceeding along with a path on RNA. In addition, when the path runs through a complementary strand sequence of sequence b, P2 binds to the sequence to act as a primer, which induces the reaction to form double stranded DNA. As a result, a promoter sequence in P1 also becomes double stranded DNA, resulting in RNA of the path sequence from sequence a to b (a reverse complementary strand sequence of original input RNA) transcripted. The output reverse complementary strand RNA is bound with P2, leading to reverse transcription. Then P1 binds to the transcripted DNA to generate double stranded DNA, and, as a result, the same RNA is output again. From the different view, in this reaction, exchange of roles between primer P1 and P2, which implement the function, allows the original primer to function as the underlying function C: Amplify (b −# a), using reverse complementary strand DNA as a argument. Also in this function, an optional sequence, P or Q, may be added to 5′- or 3′-end of the output RNA molecule as the underlying function C.

Underlying function E: Output ( ) RNA X

This function always outputs RNA of sequence X without requiring an argument.

Underlying function E is designed to always transcript RNA of sequence X without requiring an argument. This function is achieved with double stranded DNA consisting of a promoter sequence and its downstream sequence X.

Program construction with combination of the underlying functions

Combining the underlying functions above enables to construct a higher-order function. In addition, programs may also be constructed by combining above functions, arguments and return values. However, when the program showed in FIG. 5 is executed using chemical reactions, in particular, operator nucleic acids have to be designed carefully. In the above underlying functions, A, B, C and D, operator nucleic acids initiating reverse transcription at first are categorized as primer P1, and those inducing the subsequent formation of double stranded DNA are categorized as primer P2. However, primers added as substantial functions to reaction solution for the underlying function A are equal to those for B, again those for C are equal to those for D. For example, a primer pair implementing the underlying function A: Path (a→b)=>X would act as the underlying function B: path (b−# a)=>X if the primer P2 functions as a first primer. Alternatively, if the primer P1 functions as a first primer and a second primer, path (a→a)=>X may be given as the output.

Alternatively, if a promoter sequence is double stranded due to dimmer formation of primers, a wrong return value may be returned. Furthermore, when multiple functions are concurrently executed in single reaction solution, the more types of functions used, the more combinations of primers may cause interaction within a combination, resulting in chances of side reactions increased. To implement programs effectively executing targeted function reactions without the effect of side reactions, it is particularly important to consider using the combination of functions possibly having less chance of side reactions, and carefully programming and designing, in particular, a sequence of primers used in the reactions.

For example, nucleic acids including orthonormalized sequences may be used as an operator nucleic acid. The term “normalize” in “orthonormalized sequence” refers to maintain the normality of their thermal property among multiple sequences, and, in other words, make them have uniform melting temperature within certain range. The normality of the thermal property maintained, reactions would be advantageously executed using many sequences as a whole. The term “ortho” in “orthonormalized sequence” refers to give orthogonality to sequences, wherein each of all sequences included in one group of orthogonalized sequences reacts independently, and, thus, sequences included in one group of orthogonalized sequences hardly or never react among the sequences, except for desired combinations, and inside of its own sequence. In turn, a sequence included in one group of orthonormalized sequences has less or no chance to cause cross-hybridization between each sequence, and undesired hybridization inside of its own sequence.

The above orthonormalized sequences are described in H. Yshida and A. Suyama, “Solution to 3-SAT by breadth first search”, DIMACS Vol. 54 9-20(2000) and Japanese patent No. 2003-108126 in detail. Using the methods described in these references, orthonormalized sequences can be designed. Briefly, they can be produced using the method comprising: generating multiple base sequences previously in random manner: calculating the average of their melting temperature: selecting candidate sequences based on threshold limited with the average ±t° C.: and obtaining a group of orthonormalized sequences from the candidate sequences selected with an indication whether or not the sequences react independently.

The base sequences or nucleic acids included a group of orthonormalized sequences share almost similar melting temperature, have little chance to cause cross-hybridization each other and have unstable secondary structure. The orthonormalized sequences may also be used as nucleic acids of coding sequences in the following examples.

In addition, preferably, encoded nucleic acids of the invention have also orthonormalized sequences above. On the other hand, for example, total RNA purified from cells may also be used as a first encoded nucleic acids directly. In turn, without converting pre-associated information to encoded nucleic acids, the obtained nucleic acid itself (for example, a non-encoded degradable nucleic acid such as total RNA), may also be directly used as an encoded nucleic acid, regarded as information. One example is a case of using a method of the invention for gene expression analysis below. Furthermore, application of further operations to a second encoded nucleic acid obtained from former operation also enables to obtain a non-encoded single stranded nucleic acid as a return value directly. Such nucleic acids may be mRNA or adaptamer nucleic acids binding to proteins. In addition, they may be antisense RNA hybridizing to specific gene mRNA. One example is the case of using a method of the invention for intracellular molecular computing below.

In such cases, preferably, RNA used for input are allowed to react further after converted into encoded nucleic acids having orthonormalized sequences, for example, as described below.

(Gene Expression Analysis Program)

Hereinafter, the case of the application to gene expression analysis will be illustrated, as an example of programs with combination of the underlying functions above.

(Gene Encoding)

For gene analysis with DNA microarray etc, encoding techniques converting specific genes to corresponding zip codes or internal codes has been developed to control hybridization appropriately (Gerry et al. Universal DNA microarray method for multiplex detection of low abundance point mutations. J Mol Biol September 1999 17;292(2):251-62, Nishida et al. Highly specific and quantitative gene expression profiling based on DNA computing. Genome Informatics 2001 (12) 259-260, Wharam et al. Specific detection of DNA and RNA targets using a novel isothermal nucleic acid amplification assay based on the formation of a three-way junction structure. Nucleic Acids Res June 2001 1;29(11):E54-4).

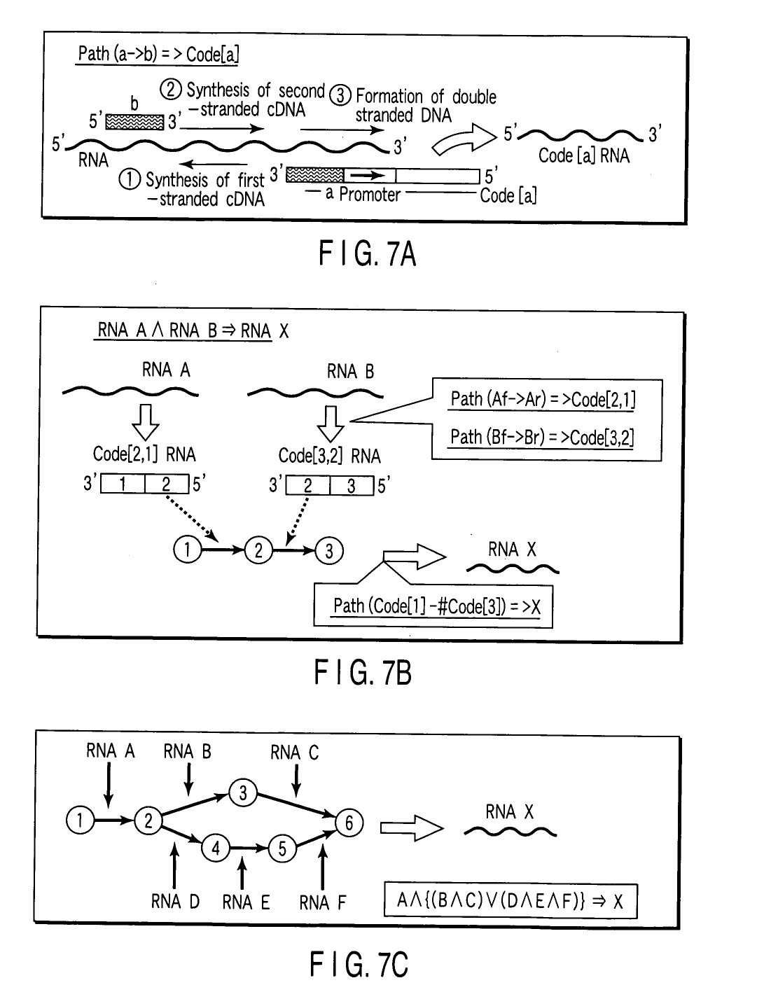

The program uses the underlying function A(path (a→b)=>X) (FIG. 7A). Sequence a and sequence b are used as a primer pair recognizing RNA of a targeted gene specifically. These sequences are incorporated at 3′-end of operator nucleic acids. Primers are designed to have incorporation of a coding sequence corresponding to sequence X of output RNA in downstream of a promoter sequence. Using such primer pairs, a function can be generated to convert an input targeted gene RNA to the corresponding coding sequence. Furthermore, using the underlying function C(Amplify) and the underlying function D(RevAmplify), it would also be possible to add sequences for labeling at 5′- or 3′-end of a partial sequence of a targeted gene.

Using the program, genes encoding can be achieved under autonomous condition. For example, it can be also applied to gene detection with DNA micro array and like. In addition, for example, a coding sequence RNA can be used as an input for an operation program with other functions to construct gene expression analysis program.

(Conversion of each Gene to a Path Element and Gene Expression Analysis with Logic Operation)

Here, a method of gene expression analysis involving encoding of each gene for a path element is described. The program example returning gene X in the presence of gene A and B is showed in FIG. 7B.

The program consists of a function converting RNA of gene A and B to coding sequences and a function recognizing a path and returning gene X. In turn, gene RNA is encoded, and the operation is carried out with the resulting encoded sequence. At first, the consideration is given to a encoding function returning coding sequence, Code[2,1], which has the sequence consisting of coding sequences, Code[2] and Code[1], aligned in the direction from 5′-end to 3′-end, in the presence of gene A using the underlying function A. In the same way, a function returning Code[3,2], which has a sequence consisting of Code[3] and Code[2] aligned, in the presence of gene B, wherein Code[1], [2] and [3] may be any sequences. Preferably, these have sequences which hardly cause mis-priming etc and have similar priming efficiency under the condition of the reaction solution. In turn, the orthonormalized sequences mentioned above are preferable.

Combining the above functions, path element Code[1]-Code[2] is formed only in the presence of gene A, and path element Code[2]→Code[3] is formed only in the presence of gene B. Therefore, only in the presence of both gene A and B, a path in RNA starting at Code[1] and ending at Code[3] is formed (FIG. 7B). Here, the underlying function B (or the underlying function A) is used to add another function returning RNA X in the presence of the path. It provides the program returning gene X only in the presence of both genes.

The key property of the method is to execute gene analysis involving conversion of each gene to each path element (1→2 and 2→3), which is a constituent of a virtual path consisting of coding sequences (in this case, path 1→2→3) and detection of the presence of the path. Extending the scale of a path and using increased types of associated genes would enable to carry out more complicated operations (FIG. 7C). Alternatively, RNA of output sequence X can be also used as an input for yet another path to make paths multilayered.

(Gene Expression Analysis using Neural Networks)

In gene expression analysis with logic operation, gene expression patterns have to be known. In addition, essentially, it analyzes only existence of genes and can not estimate information of the concentration. A neural network constructed using an information processing method of the invention will be illustrated to show an example of methods also enabling estimation of concentration of genes whose expression patterns are unknown.

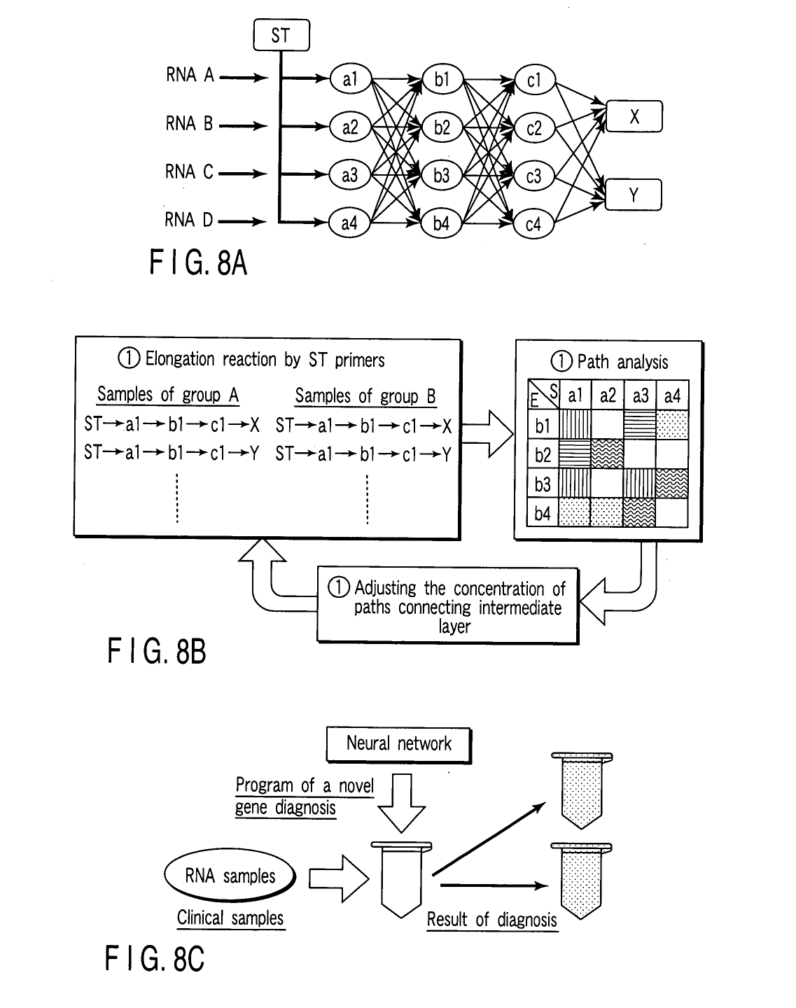

Some scientists have proposed ideas to apply a neural network constructed with a DNA computer to gene expression analysis (Mills Gene expression profiling diagnosis through DNA molecular computation. Trends Biotechnol April 2002; 20(4):137-40). However, it was difficult to carry out complicated analysis using conventional ideas because it was a single-layered simple perceptron model without intermediate layers. In addition, it required a manipulation containing multiple steps. On the contrary, using an information processing method of the invention, multilayered perceptron which may execute a complicated analysis can be achieved in autonomously working reaction system (FIG. 8A).

At first, genes are encoded to carry out gene analysis. The encoding function is made to output Code[a1,ST] in the presence of RNA A. This may be associated to path ST→a1. Similar functions are also configured for RNA B, C and D to replace them into path ST→a2, a3 and a4 respectively. These encoding functions carry out input into a neural network depending on the existence of each gene RNA. All path units: a1→b1, a1→b2, a1→b3, . . . , b4→c4 and c1→X, c1→Y, c2→X, . . . , c4→Y, which connect intermediate layers of perceptron, can be generated by the corresponding RNA output using the underlying function E: Output( ). In addition, using the underlying function B, a program is constructed with introduction of a function returning x depending on the existence of path ST→X (path (ST−# X) x) and a function returning y depending on the existence of path ST→Y (Path (ST−# Y) y). As a result, a neural network is formed to change the proportion of output x to y depending on input RNA is formed (FIG. 8A). It is possible to change accordingly the number of input layers, intermediate layers and output layers. In addition, the intensity of each RNA path may be controlled by adjusting the concentration of the corresponding Output( ) function.

Using the method showed in FIG. 8B, a learning process may be achieved for a neural network. Specifically, at first, RNA of the samples of group A and B are given as inputs to reaction solution containing functions relating to paths connecting inputs and intermediate layers, and ST primers to initiate elongation reaction of ST primers. In each reaction solution, depending on the situations of given input RNA and paths for intermediate layers, each path, starting at ST and ending at X or Y, is reverse transcripted, which provides corresponding cDNA synthesized ((1)). Then, the paths are analyzed, divided depending on terminal sequence of the resulting ST primer elongation product, which is either X or Y. Intermediate paths in group A and B, (a1→b1, a1-b2, . . . ), are compared each other in regard to their concentration ((2)). It is expected that this job is performed by real-time PCR and DNA microarray method, for example, using samples containing complexed paths. Based on this result, concentration of Output( ) function is adjusted to intensify desired paths ((3)). For example, when it is desired to relate group A and B to output x and y respectively, comparison is made between sample group A-X ending path and sample group B-Y ending path, and between sample group A-Y ending path and sample group B-X ending path to increase the path units specific to the former and decrease those specific to the latter. Learning can be achieved by repeating this cycle, (1)→(2)→(3)

Utilizing of gene expression analysis technique involving the neural network of this molecular computer may provide a novel gene diagnosis technique (FIG. 8C). For example, the reaction solution is prepared to contain operator nuclei acids necessary for above reactions. Then, RNA obtained from a clinical sample is added to the reaction solution to initiate the reactions. Constructing the programs to give given outputs when given genes are expressed in given combination, it would be possible to analyze gene expression pattern and level easily.

(Extension of Functions)

Usable Functions for the invention are not limited to above 5 functions. It is possible to define various functions using various operator nucleic acids.

For example, in all of above underlying functions, which is constructed to lead reverse transcription reaction initiated with P1 and hybridization of P2 with cDNA generated from the reverse transcription, P2 may also be used as a primer for RNA. In this case, 3′-end of P2 would be changed through elongation reaction. Such a change of 3′-end sequence may be considered to correspond to the change of detail of a function. The use of such a change enables to extend the concept of functions. In addition, achieving the chemical reactions exemplified below in the hardware reaction solution, it would be possible to extend the definitions of functions available for programs beyond 5 underlying functions.

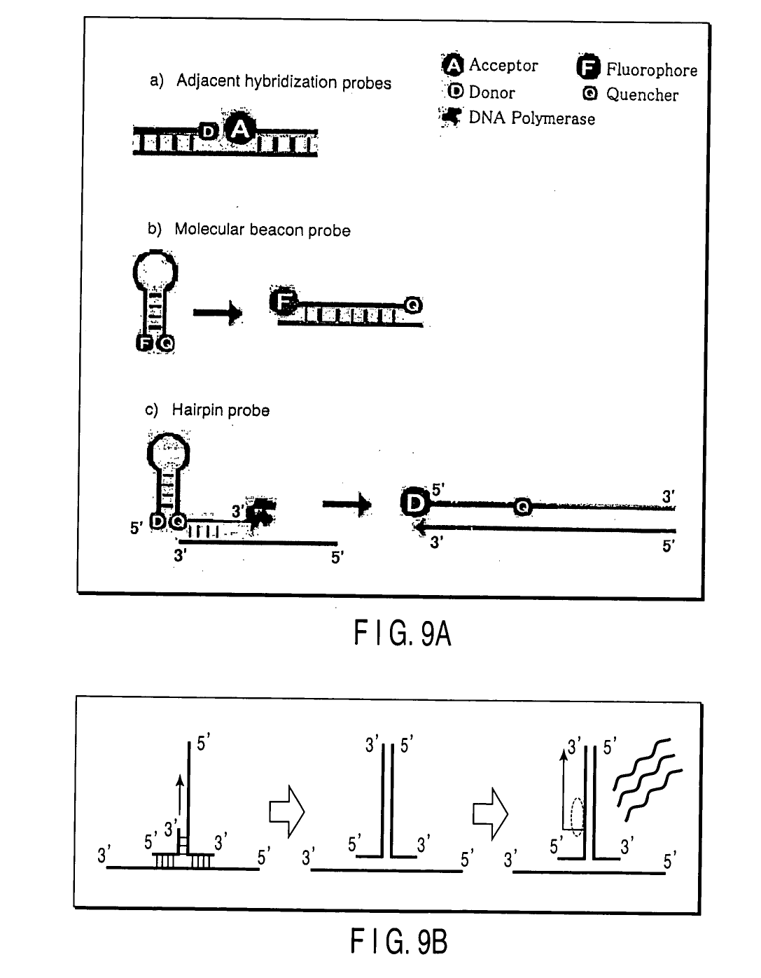

In order to return a result of a program, as a computer, it is necessary to detect the output resulted from a series of reactions corresponding to functions. A program consisting of only above underlying functions, all of which return RNA as return values, also give RNA molecules as final outputs. These output RNA can be purified with molecular biology procedures. The use of techniques such as RT-PCR, northern blotting and DNA microarray also enables to detect output RNA. Taking the advantages of an autonomously workable molecular computer of the invention, it would be more effective to carry out a series of steps leading up to the detection of results in single reaction solution. Therefore, it is preferable to detect output RNA molecules in the computing reaction solution directly. For example, it is possible to apply Fluorescence Resonance Energy Transfer (FRET) technology to detect RNA molecules directly. FRET is very useful to detect fluorescence externally to take information. FRET technology has been applied to real-time PCR with fluorescence labeled DNA probes (Didenko, DNA probes using fluorescence resonance energy transfer (FRET): designs and applications. Biotechniques November 2001; 31(5):1106-16, 1118, 1120-1). For example, the use of FRET probes showed in FIG. 9A enables to apply it as fluorescence outputting functions to output of molecular computers. Adjacent hybridization probes and Molecular beacon probe have a property to return fluorescence in the presence of specific targeted sequences, thus they may be directly used as output detecting functions of a molecular computer (FIGS. 9A-a, b). Furthermore, Hairpin probe produces fluorescence when primers are double stranded through DNA elongation reaction (FIGS. 9A-c), and thus the use of primers with such a structure as a substitute for primer P1 in the underlying function A or primer P2 in the underlying function B enables to configure the function returning fluorescence only in the presence of an appropriate path.

Using these fluorescence outputting functions, it is possible to design gene diagnosis program making it possible to carry out the course leading up to detection of output in single step. For example, in a gene expression analysis using a neural network showed in FIG. 8C, wherein the results are given by comparison of concentration between x and y, outputs can be detected with different probes made with different fluorochromes to recognize x and y respectively. Alternatively, the fluorescence outputting function, “the function returning fluorescence in the presence of path ST→X”, involving Hairpin probes, may also be constructed instead of the function, “the function returning x in the presence of path ST→X. Assigning a different fluorescence to each final output, it would be possible to detect output through the comparison of their fluorescence intensity.

For further reactions, other type of primers may be used, for example, based on 3-way junction (3WJ) structure, published by Wharam et al., in 2001, (FIG. 9B). This is a primer contributing expression of RNA having specific sequence in the presence of a targeted sequence. This primer can be also applied to an information processing method of the invention because the reaction may occur in the presence of DNA dependent DNA polymerase activity and DNA dependent RNA polymerase activity. In particular, it can be used for gene encoding reactions.

Furthermore, in terms of extension of functions, for example, RNA output from certain function may also be used for an operation with functions. For example, RNA molecules themselves output from each function, which may act as primers, may be allowed to act as operator nucleic acids in an operation with functions.

Furthermore, ribozymes have been studied to utilize as elements for molecular computers (Wickiser et al. Oligonucleotide Sensitive Hammerhead Ribozymes As Logic Gates. Eighth International Meeting on DNA Based Computers, June 2002 10-13; Hokkaido University, Japan). Ribozymes are known as RNA molecules having enzyme activity. When such ribozymes are used, RNA molecules themselves, which are generated as outputs in functions, may be act as ribozymes, resulting in an output RNA fulfilling a new feature as a function directly. Such ribozymes may be used as functions used in an information processing method of the invention.

Executing reactions other than the above exemplified underlying functions in hardware of the molecular computer would provide further function enhancement of the computer.

As described above, Combination of 4 types of reactions, RNA dependent DNA polymerase activity, DNA dependent DNA polymerase activity, DNA dependent RNA polymerase activity and RNaseH, which are critical reaction activity for retrovirus genome amplification, provides an autonomous running programmable molecular computer.

Specifically, a computer characterized by consisting of containers containing operator nucleic acids for carrying out operations with desired functions, a suitable reaction solution and suitable enzymes is provided as a molecular computer for carrying out the operation with the information processing method described above. Although 5 types of underlying functions are expediently defined as functions constituting a program in a molecular computer, more generally, the following 3 kinds of oligo nucleic acids are added to hardware of a molecular computer as programs; a nucleic acid containing a promoter placed in 5′-end direction, a nucleic acid containing a promoter placed in 3′-end direction and a nucleic acid without a promoter sequence. In turn, it can be said to be a system in which elongation reaction is initiated appropriately if RNA given as an input to a reaction system containing these oligo nucleic acids, and when a promoter sequence is made double stranded at any site, RNA of the downstream sequence is returned.

On the other hand, the usable containers for a molecular computer include, for example, sample tubes, test tubes and micro channels conventionally used for nucleic acid reactions. In addition, single container is enough for the molecular computer, but plural containers may be used.

If cells or tissues are used as containers, desired gene transcription can be also controlled depending on the results from autonomous detection of gene expression level and pattern in the living cells. Therefore, output of RNA can be controlled in living cells, which will provide a new controlling mechanism of cells. For example, specific genes can be expressed only in cells in which genes are expressed in specific pattern, and, the genes normalizing cells can be also expressed only in targeted cells, such as cancer cells. Such techniques may be applied to techniques such as gene therapy.

To carry out information processing with an information processing method of the invention, necessary operator nucleic acids may also be provided as a kit. The kit contains operator nucleic acids for carrying out operations with desired functions. Preferably, the kit contains an operator nucleic acid comprising one or more sequences selected from sequences acting as a primer for a first single stranded nucleic acid, promoter sequences and sequences acting as a primer for any nucleic acid.

In addition, the kit may contain not only an operator nucleic acid but also a suitable reaction solution and suitable enzymes. Suitable reaction solution include, for example, buffers suitable for a synthesis reaction, an amplification reaction, a reverse transcription reaction, a transcription reaction and a degrading reaction, and suitable enzymes include, for example, enzymes having DNA dependent DNA polymerase activity, those having RNA dependent DNA polymerase activity, those having DNA dependent RNA polymerase activity and RNaseH.

When the kit described above is a kit for gene expression analysis, for example, as described in the above section “gene expression program”, it would contain operator nucleic acids necessary for encoding, enzymes having DNA dependent DNA polymerase activity, those having RNA dependent DNA polymerase activity, those having DNA dependent RNA polymerase activity and RNase H as well as a suitable reaction solution, 40 mM Tris-HCl (pH 8.0), 50 mM NaCl, 8 mM MgCl₂, 5 mM DTT. Above enzymes may be pre-added in a reaction solution. For example, the kit may be used as follows: a RNA sample is added to a buffer solution containing all of enzymes at 50° C. and mixed well, then the reaction mixture is incubated at 50° C. For example, 3 μl of enzyme buffer is added per tube in total volume of 25 μl, which is allowed to react for 30 min.

The reaction required for execution of programs are substantially same as the reactions actually caused by retrovirus and retrotransposon in living cells, suggesting the possibility for achievement of a molecular computer with the system in living cells. When this intracellular molecular computing is materialized, for example, the gene expression analysis program in living cells combined with fluorescence outputting functions, it can be also applied to the technology for nondisruptive external monitoring of the gene expression pattern in living cells.

Alternatively, outputting RNA of gene which controls cellular activity also provides the program which controls cellular activity depending on gene patterns. For example, gene therapy may also be achieved to involve expression of introduced specific genes only in defective cells by input of marker genes for a disease such as cancer.

(Advantageous Effect of the Invention)

A programmable autonomous running molecular computer can be generated by using an information processing method of the invention. Such a computer has versatility to execute different programs in single hardware. In particular, it can be applied to uses such as research and development regarding function analysis of genes, gene diagnosis and like, for which the needs may grow in the future.

Gene-expression-analysis executing programs based on logic operation or neural network combined with fluorescence outputting functions, it may be allowed to carry out autonomously all of measurements and analysis of genes, and output of the results. Furthermore, using the method involving above neural network, it would be possible to analyze gene expression in principle even if relationship between gene expression pattern and phenotypes is not clear. In addition, it is-also possible to estimate information about concentration of expressed genes.

### EXAMPLE

(Materials and Methods)

(Equipments and Reagents)

Double-stranded DNA molecules were detected with Agilent 2100 bioanalyzer (Agilent Technologies) after electrophoresis. Reagents used in practice of the method are DNA 500 LabChip® kits or DNA 7500 LabChip® kits. Real-time PCR was carried out using LightCycler™ Quick System 330 (Roche Diagnostics Co.). Reagents used for the PCR were LightCycler™ FastStart DNA Master SYBR® Green I, purchased from said company. Preparation of reagents and operation of instruments were carried out according to manufacturer's manuals.

(Design of Gene Specific Sequences)

Primers recognizing TGTP gene and Vitronectin gene were designed respectively using the specific primer design program developed by Takashi Mishima et al. (“Study for a probe and primer sequence design method for measurement of gene expression in large scale”, Graduate School of Science, The University of Tokyo, master's thesis 2001), Primer3 (Rozen and Skaletsky, Primer3 on the WWW for general users and for biologist programmers Methods Mol Biol 2000; 132:365-86) available to the public as a primer design software, and like others, and suitable primers are selected from the generated primers.

The used sequences specific to TGTP gene and Vitronectin gene are summarized below (numbers in parentheses denotes the location of a primer in a RNA molecule of either TGTP or Vitronectin. “S” refers to a sense strand sequence, “A” refers to an anti strand sequence.)

| | |
|---|---|
| Sequence name | Sequence |

TGTP specific primer sequences

| | | |
|---|---|---|
| TGTP-S1 | 5′- CAGATATATATGGTCCCACC -3′ (1302, A) | (SEQ ID NO:1) |
| TGTP-S2 | 5′- ACTTACTATCGCATGGCTTA -3′ (1201, S) | (SEQ ID NO:2) |
| TGTP-AF | 5′- CAGGATTTGAACATGTCTGTGGAT -3′ (1051, S) | (SEQ ID NO:3) |
| TGTP-AR | 5′- GCTTGTCTTCTAAGGACTCATCATTG -3′ (1119, A) | (SEQ ID NO:4) |
| TGTP-PS | 5′- GGGGATGAATTTCTACTTTG -3′ (582, S) | (SEQ ID NO:5) |
| TGTP-PE | 5′- AGAGTGAACACTGATTGGAA -3′ (1364, A) | (SEQ ID NO:6) |

Vitronectin specific primer sequences

| | | |
|---|---|---|
| Vitronectin-P1 | 5′- TTTGTCTCCAGAGAAGAAAT -3′ (1313, A) | (SEQ ID NO:7) |
| Vitronectin-P2 | 5′- GCTAGGAACCTACAACAACT -3′ (1236, S) | (SEQ ID NO:8) |
| Vitronectin-PS | 5′- GTACCCCAAACTTATCCAAG -3′ (570, A) | (SEQ ID NO:9) |
| Vitronectin-PE | 5′- GTAGGGAGGATTCACAGAGT -3′ (1367, S) | (SEQ ID NO:10) |

If required, to above sequences are added a promoter sequence or a coding sequence at their 5′-end to use for the study. Synthesis of primer DNAs having less than 30 bases were basically customized by Oligo Japan Co. as Easy oligos®. Longer primers, having 30 bases or more, were customized by Sawady Technology Co. Ltd.

(Design of Coding Sequences)

Oligo DNAs containing an artificially generated “coding sequence” were used in this example. A “coding sequence”, as used herein, refers to a sequence pair of which members have the same base length and are designed to be characterized by having the equalized melting temperature of double stranded DNA with calculation using the nearest-neighbor method (SantaLucia A unified view of polymer, dumbbell, and oligonucleotide DNA nearest-neighbor thermodynamics. Proc Natl Acad Sci USA February 1998 17;95(4):1460-5) and having little chance of the formation of stable secondary structure and mis-hybridization (Yoshida et al “Solution to 3-SAT by breadth first search. DIMACS Series in Discrete Mathematics and Theoretical Computer Science, 2000 54: 9-22, American Mathematical Society). In the study, the following 5 sequences, having 25-base length were used.

| | |
|---|---|
| Sequence | |
| name | Sequence |

| | | |
|---|---|---|
| Code [1] | 5′- TGAAGTCACCACAACACACAGTACA -3′ | (SEQ ID NO:11) |
| Code [2] | 5′- GACAAACACCCCGAATACAAACAGC -3′ | (SEQ ID NO:12) |
| Code [3] | 5′- AGTATCGAAGCGTGTGTCTGAAGAT -3′ | (SEQ ID NO:13) |
| Code [4] | 5′- CAAAAGAGTTAGGATGGGAGCTGGA -3′ | (SEQ ID NO:14) |
| Code [5] | 5′- TCGATATGGGTGGTACATGAGAGGT -3′ | (SEQ ID NO:15) |
| Code [6] | 5′- CTCCGCTCCTCTATTCATTCCCTAG -3′ | (SEQ ID NO:16) |

(Primer Sequences Used for the Computing Reaction)

Gene specific sequences, coding sequences and the T7 promoter sequence etc were combined to make specialized oligo DNAs for using the computing reaction. Their names, structures and sequences are listed below. (In the item “Structure”, [ ] refers to a sequence name of gene specific sequence, <>refers to a sequence name of coding sequence, {T7} refers to the T7 promoter sequence. Tg is a sequence having 6-base length, sequence 5′-GGGAGA-3′, Tc is a 9-base length sequence, 5′-ATAGGGAGA-3′. a′ headed sequences denote reverse complementary stranded sequences. S denotes other sequences.

Name: TGTP-P1
Structure: 5-S-a{T7}-[TGTP-S1]-3′

(SEQ ID NO:17)

Sequence: 5′- CTGAGGTTATCTTGGTCTGGGGAGATCTCCCTATAG
TGAGTCGTATTA CTGAGGTTATCTTGGTCTGGGG AGACAGATATATAT
GGTCCCACC -3′
Name: TGTP-T21
Structure: 5′-a<Code[1]>-Tg-a<Code[2]>-a{T7}-
[TGTP-S1]-3′

(SEQ ID NO:18)

Sequence: 5′- TGTACTGTGTGTTGTGGTGACTTCA TCTCCCGCTG
TTTGTATTCGGGGTGTTTGTC TCTCCCTATAGTG AGTCGTATTACAGA
TATATATGGTCCCACC -3′
Name: Vitronectin-T32
Structure: 5′-a<Code[2]>-Tg-a<Code[3]>-a{T7 }-
[Vitronectin-P1]-3′

(SEQ ID NO:19)

Sequence: 5′- GCTGTTTGTATTCGGGGTGTTTGTC TCTCCCATCT
TCAGACACACGCTTCGATACT TCTCCCTATAGTG AGTCGTATTATTTG
TCTCCAGAGAAGAAAT -3′
Name: aT21
Structure: 5′-Tc-<Code[2]>-aTg-<Code[1]>-3′

(SEQ ID NO:20)

Sequence: 5′- ATAGGGAGA GACAAACACCCCGAATACAAACAGCG
GGAGA TGAAGTCACCACAACACACAGTACA -3′

Comment: Complementary sequence to 5′-end of

TGTP-T21 primer
Name: aT32
Structure: 5′-Tc-<Code[3]>-aTg-<Code[2]>-3′

(SEQ ID NO:21)

Sequence: 5′- ATAGGGAGA AGTATCGAAGCGTGTGTCTGAAGATG
GGAGA GACAAACACCCCGAATACAAACAGC -3′

Comment: Complementary sequence to 5′-end of Vitronectin-T32 primer

Name: TGTP-PT
Structure: 5′-“GATGCA”{T7}-[TGTP-PS]-3′

(SEQ ID NO:22)

Sequence: 5′-GATGCA TAATACGACTCACTATAGGGAGAGGGGATG
AATTTCTACTTTG-3′

Comment: A primer used for in vitro synthesis of TGTP gene. The sequence of first 20 bases at the 3′-end is identical with the sequence of 5′-end of synthesized TGTP RNA molecule.

Name: Vitronectin-PT
Structure: 5′-“GATGCA”-{T7}-[ Vitronectin-PS]-3′

(SEQ ID NO:23)

Sequence: 5′- GATGCA TAATACGACTCACTATAGGGAGAGTACCC
CAAACTTATCCAAG -3′

Comment: A primer used for in vitro synthesis of Vitronectin gene. The sequence of first 20 bases at the 3′-end is identical with the sequence of 5′-end of synthesized Vitronectin RNA molecule.

Name:20/aCode [1]

(SEQ ID NO:24)

Sequence: 5′- TGTACTGTGTGTTGTGGTGA -3′

Comment: This is the first 20 bases at 5′-end of Code[1] sequence, and its Tm value is approximately 48° C. in the computing reaction solution.

Name: aC3-T45
Structure: 5′- a<Code[5]>-aTg-a<Code[4]>-a{T7}-Tg-
<Code[3]>-3′

(SEQ ID NO:25)

Sequence: 5′- ACCTCTCATGTACCACCCATATCGA TCTCCCTCCA
GCTCCCATCCTAACTCTTTTG TCTCCCTATAGTGAGTCGTATTAGGGAG
A AGTATCGAAGCGTGTGTCTGAAGAT -3′
Name: aT45
Structure: 5′- Tc-<Code[4]>-aTg-<Code[5]>-3′

(SEQ ID NO:26)

Sequence: 5′- ATAGGGAGA CAAAAGAGTTAGGATGGGAGCTGGAG
GGAGA TCGATATGGGTGGTACATGAGAGGT -3′

Comment:Complementary sequence to 5′-end of aC3-T45.

Name: aC3-T465
Structure: 5′- a<Code[5]>- aTg-a<Code[6]>-aTg-
a<Code[4]>-a{T7}-Tg-<Code[3]>-3′

(SEQ ID NO:27)

Sequence: 5′- ACCTCTCATGTACCACCCATATCGA TCTCCCCTAG
GGAATGAATACAGGAGCGGAG TCTCCCTCCAGCTCCCATCCTAACTCTT
TTG TCTCCCTATAGTGAGTCGTATTAGGGAGA AGTATCGAAGCGTCTG
TCTGAAGAT -3′
Name: aT465
Structure: 5′- Tc-<Code[4]>-aTg-<Code[6]>-aTg-
<Code[5]>-3′

(SEQ ID NO:28)

Sequence: 5′- ATAGGGAGA CAAAAGAGTTAGGATGGGAGCTGGAG
GGAGA CTCCGCTCCTCTATTCATTCCCTAG GGGAGATCGATATGGGTG
GTACATGAGAGGT -3′

Comment: Complementary sequence to 5′-end of aC3-T465.

(Preparation of RNA Samples)

TGTP and Vitronectin RNA molecules, as well as Code[2,1] and Code[3,2] RNA molecules used for the computing reaction were prepared with an in vitro transcription method.

TGTP gene and Vitronectin gene were prepared with the following procedures. The graft versus host reaction (GVHR) is induced in BALB/c mice by implantation of spleen cells derived from C57/BL10 mice. C57/BL10 mice derived spleen cells were given by Prof. Katsushi Tokunaga, Faculty of Medicine, The University of Tokyo. Then, total RNA is prepared from a liver taken from the mice 2 days after the implantation. An equivalent of this sample has been confirmed to contain RNA of TGTP gene and Vitronectin gene by a semiquantitaive real-time PCR method (Wakui et al. 2001). Then, reverse transcription was performed using total RNA as a template to generate cDNAs of TGTP gene and Vitronectin gene. TGTP-PE and Vitronectin-PE were used as primes in the reverse transcription for TGTP gene and Vitronectin gene respectively. AMV Reverse Transcriptase XL, containing 50 mM Tris-HCl (pH 8.3), 4 mM DDT, 10 mM MgCl₂, 100 mM KCl, 0.5 mM dNTPs, 800 nM of each primer and 0.3 Units/μl, (Takara Bio Inc.), was used as a reaction solution for the reverse transcription, to which total RNA was added when the reaction performed. The hot-start method was used to perform the reaction. Specifically, 9.5 μl of the reaction solution without an enzyme was incubated for 5 minutes at 65° C., followed by 3 μl of a solution containing the enzyme added. After the solution added the enzyme was incubated for 60 min at 50° C., 0.5 μl of Ribonuclease H (2 U/l; Invitrogen) was added, and then the mixture was reacted for 20 min at 37° C. Then, PCR reaction was separately performed using resulting cDNA as a template. When the reactions were carried out, the pair of TGTP-PE and TGTP-PT and the pair of Vitronectin-PE and Vnct-PT were used as primer pairs for TGTP and Vitronectin respectively, wherein TGTP-PT primer and Vitronectin-PT primer are oligo DNAs added a clump sequence having 6-base length (5′-GATGCA-3′ (SEQ ID NO:26)) and T7 promoter sequence having 23-base length (5′-TAATACGACTCACTATAGGGAG A-3′(SEQ ID NO:27)) at 5′-end of gene specific sequences, TGTP-PS and Vitronectin-PS respectively. TaKaRa Ex Taq™ (Takara Bio Inc.) was used in the PCR reaction, which was performed following the attached protocol (Cool start method). Briefly, the solution, prepared by adding 0.8 μl of each primer DNA, each of 0.2 mM dNTPs, 40 U/ml enzyme and 1 μl of cDNA sample to 25 μl of the reaction buffer, was applied to the reaction for 31 cycles of 94° C.-30 sec, 60° C.-90 sec and 72° C.-60 sec, followed by 720 ° C.-10 min. Detection of actually resulting PCR products with electrophoresis revealed that this reaction provided single bands having the same base length as each expected value, which were 831-base and 846-base double stranded DNA for TGTP and Vitronectin respectively (data not shown).

In vitro transcriptions were performed separately using T7 promoter-containing double stranded DNA of either TGTP or Vitronectin gene, generated from above PCR reaction, to produce RNA molecule for each gene. This reaction, for each genes, was carried out with 100 μl of the reaction solution aliquoted into 4 tubes, in each of which 500 U/ml T7 RNA Polymerase (Invitrogen) and 1μl of double stranded template were added to the reaction buffer, comprising of 40 mM Tris-HCl (pH 8.0), 8 mM MgCl₂, 2 mM Spermidine-(HCl)₃, 25 mM NaCl, 5 mM DDT, 0.4 mM NTPs. After incubation for 1 hr at 37° C., to the mixture was added 2.5 μl of 1 U/μl Deoxyribonuclease I (Amplification Grade; Invitrogen) and incubated for further 15 min at 37° C. The resulting reaction products were purified with ethanol precipitation. The ethanol precipitation was performed with Pellet Paint® Co-Precipitant (Novagen) following the attached protocol. The resulting precipitates were solved in DEPC water to store at −20° C. before use.

Code[2,1] and Code[3,2] RNA molecules were in vitro synthesized using customized oligo DNAs, TGTP-T21 and Vitronectin-T32. For each reactions, 20-base length primers complementary to 3′-end of the oligo DNAs are mixed with the PCR reaction solution and incubated for 5 minutes at 94° C., then added buffer containing the enzyme at 80° C., followed by incubation for 5 minutes at 60° C. and then 72° C. for 60 minutes. The resulting double stranded DNA containing T7 promoter sequence was used for in vitro transcription to produce coding RNA. The transcription reaction, Deoxyribonuclease I treatment and ethanol precipitation method were similar to the case of TGTP gene and Vitronectin gene.

(Computing Reaction)

Computing reaction executing various function reactions with DNA primers are accomplished by coexisting of an enzyme with RNA dependent DNA polymerase activity, an enzyme with DNA dependent DNA polymerase activity or an enzyme with DNA dependent RNA polymerase activity in single buffer, in which the enzymes can be active. The reaction solution comprises 40 mM Tris-HCl (pH 8.0), 50 mM NaCl, 8 mM MgCl₂, 5 mM DTT and 0.3 U/μl AMV Reverse Transcriptase XL (Takara Bio Inc.), 0.04 U/μl Ex Taq™ (Takara Bio Inc.), 3.2 U/μl Thermo T7 RNA Polymerase (TOYOBO). To this reaction solution are added DNA primers and a RNA template accordingly. Unless otherwise provided, DNA primers are added in the final concentration of 1 nM. The reaction was carried out with the hot start method, wherein the reaction solution without enzymes was incubated at 65° C. for 5 minutes. Then, the buffered solution containing all enzymes was added at 50° C. and mixed well, followed by incubation at 50° C. Unless otherwise provided, 3 μl of enzyme buffer is added per tube in 25 μl of total volume of the reaction solution and allowed to react for 30 min. The reaction mixture was incubated at 85° C. for 10 minutes to deactivate the transcription enzyme immediately after completion of the reaction.

(Detection of RNA Products After the Computing Reaction)

The RNA products resulted from the computing reaction were detected by reverse transcriptional-PCR after DNA degraded with enzymes.

Before degrading of DNA with enzyme, column purifying was performed to remove enzymes potentially binding to DNA and inhibiting the enzyme degrading, such as a Taq polymerase. After a sample solution was prepared by addition of DEPC treated water to the computing reaction solution to 50 μl total volume, it was charged to a MW cut off: 10,000-column, MICROCON YM-100 (Millipore) and centrifuged at 4° C., 12,000 rcf for 10 minutes. Collected flow-through solution was applied to MICROCON YM-10 (Millipore, molecular weight cut off=10,000) again and centrifuged at 4° C., 12,000 rcf for 50 minutes, followed by centrifugation of the column placed upside down in a new tube at 4° C., 12,000 rcf for 10 minutes to collect concentrated solution remaining on upper side of the column.

DNA degrading reaction was performed at room temperature for 15 minutes in 10 μl of the reaction solution which was prepared by addition of 1 μl of each sample collected from above to 20 mM Tris-HCl (pH 8.4), 2 mM MgCl₂, 50 mM KCl and 0.1 U/μl Deoxyribonuclease I (Amplification Grade; Invitrogen). After the reaction, to the reaction solution was added 1 μl of 25 mM EDTA and then incubated at 65° C. for 10 minutes.

Reverse transcription reaction was performed in 12.5 μl of a reaction solution per tube, which was prepared by addition of primer DNAs in final concentration of 600 mM and 1 μl of a DNase I reaction product obtained above to 50 mM Tris-HCl (pH 8.3), 4 mM DDT, 10 mM MgCl₂, 100 mM KCl, 0.5 mM dNTPs and 0.3 Units/μl AMV Reverse Transcriptase XL (Takara Bio Inc.). This reaction was carried out with the hot start method, wherein the solution comprising all component except for the enzyme was incubated at 65° C. for 5 minutes, followed by 3 μl of the buffered solution with the enzyme added at 50° C. Then, it was allowed to react at 50° C. for 1 hr, followed by 94° C. for 10 minutes.

Resulting cDNA was quantitatively analyzed by real-time PCR. To 20 μl of reaction solution, prepared following the manufacturer's manual, was added 1 μl of the reverse transcriptional product and incubated at 94° C. for 10 minutes, and then PCR reaction was performed. The PCR reaction was carried out for 40 cycles of 94° C.-3 sec, 60° C.-10 sec and 72° C.-5 sec to amplify a coding sequence and gene sequence with less than 300 base length, and for 40 cycles of 94° C.-25 sec, 60° C.-10 sec, 72° C.-25 sec to amplify a gene sequence with 300 bases or more. The quantitative concentration analysis was performed by comparing PCR amplification curves obtained above to those from simultaneous PCR reactions with single stranded DNA in finale concentrations of 0.1 nM, 0.03 nM, 0.01 nM, using the software appended to a machine. In addition, the PCR reaction was stopped at an appropriate time point to take halfway amplified samples, which were detected and analyzed by gel electrophoresis using Agilent 2100 bioanalyzer (Agilent Technologies).

(Detection of Intermediate DNA Products in a Computing Reaction)

Intermediate products comprising single stranded and double stranded DNA generated in the reaction solution were detected to confirm the progress of the computing reaction. Single stranded or double stranded DNAs generated by reverse transcription reaction were detected with amplifying them by PCR reaction after purification of them. DEPC treated water was added to the computing reaction solution to adjust the sample volume to 50 μl, which was pipetted into a column, MICROCON YM-100 (Millipore) (MW cutoff value is 100,000) and centrifuged at 4° C., 12000 rcf for 10 minutes. Flow-through solution from the column was collected and pipetted into MICROCON YM-100 (Millipore) (MW cutoff value is 100,000), which was centrifuged 4° C., 12000 rcf for 50 minute, followed by further centrifugation of the column placed upside down in a new tube at 4° C., 12000 rcf for 10 minutes to collect concentrated solution remaining at upper side of the column. The resulting solution was used for PCR to amplify single stranded DNAs in the reaction solution containing buffer added appropriate primers and Ex Taq® (Takara Bio Inc.). The amplification was carried out with the cool start method, for 31 cycles of 94° C.-30 sec, 60° C.-60 sec and 72° C.-60 sec, followed by incubation at 72° C. for 10 minutes. Resulting amplified products were detected by gel electrophoresis.

Double stranded DNA generated from the DNA double-strand formation reaction was detected by gel electrophoresis using Agilent 2100 bioanalyzer (Agilent Technologies). Base length and concentration of double stranded DNA were determined following the protocols of the instrument.

Results

(Development of Hardware)

To achieve a molecular computer simulating retrovirus genome amplification reactions, at first, the condition of the reaction solution was considered to generate all chemical reactions required for a molecular computer. This reaction solution is critical because it acts as hardware constructing the molecular computer.

For the hardware used here, it is necessary to allow all enzymes, which have DNA dependent DNA polymerase activity, RNA dependent DNA polymerase activity, DNA dependent RNA polymerase activity and RNaseH activity respectively, to be active simultaneously in single tube maintained at the certain temperature. We performed the experiment following the condition used in 3SR amplification technique (Guatelli et al. Isothermal, in vitro amplification of nucleic acids by a multienzyme reaction modeled after retroviral replication. Proc Natl Acad Sci USA October 1990; 87(19):7797), in which the similar reaction solution has been achieved. However, when the experiment was carried out following the conditions for 3SR, wherein, as well as in the similar technique, the reaction temperature is lower, 37° C. to 42° C., than annealing temperature in PCR reaction, it showed the difficulty to allow primer DNAs used for reverse transcription and DNA double-strand formation reaction to act specifically, resulting in causing more frequent dimmer formation of having no targets particularly, as well as inhibition of expected reactions by non-specific reactions (data not shown).

Such properties are not suitable for the hardware of the molecular computer, in which DNAs are used for input of programs, thus we considered setting the reaction temperature higher to achieve highly specific priming. AMV reverse transcriptase, T7 RNA polymerase and RNaseH were used in the 3SR, however two latter enzymes would be inactivated at higher reaction temperature. Therefore, Thermo T7 RNA Polymerase (TT7; TOYOBO) and Thermus thermophilus RibonucleaseH (Tth RNaseH; TOYOBO) were examined for the use as an enzyme showing DNA dependent RNA polymerase activity and one showing RNaseH activity respectively at higher reaction temperature. AMV reverse transcriptase, TT7 and Tth RNase have been confirmed to be active below 65° C., 50° C. and 90° C. respectively, and at as low as approximately 37° C. Preferably, this experiment was performed as high temperature as possible, thus, the reaction was examined at 50° C. or higher.

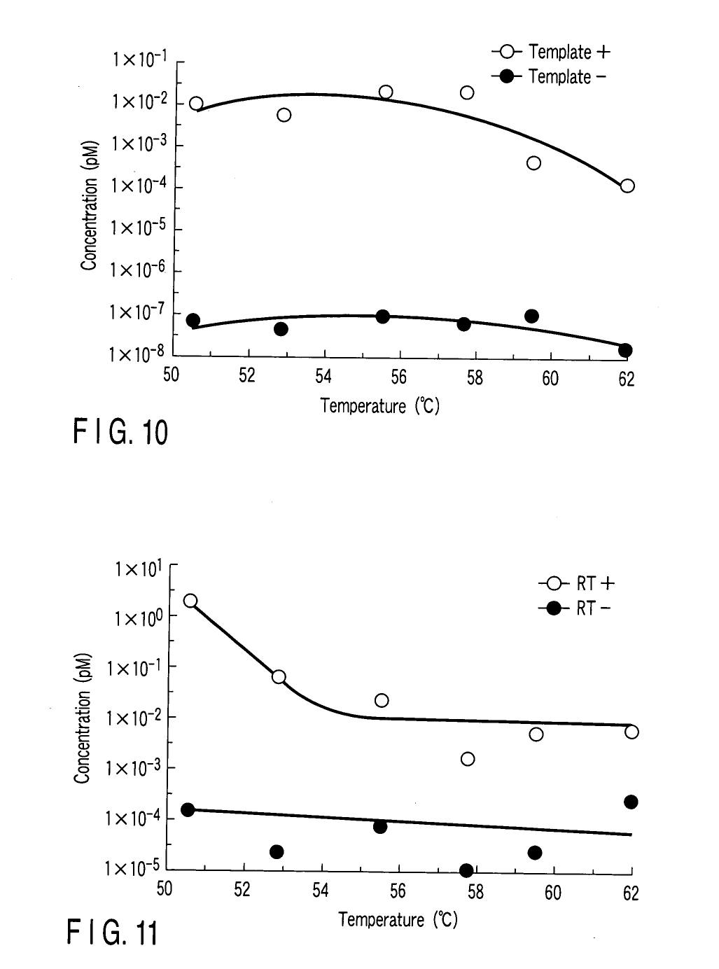

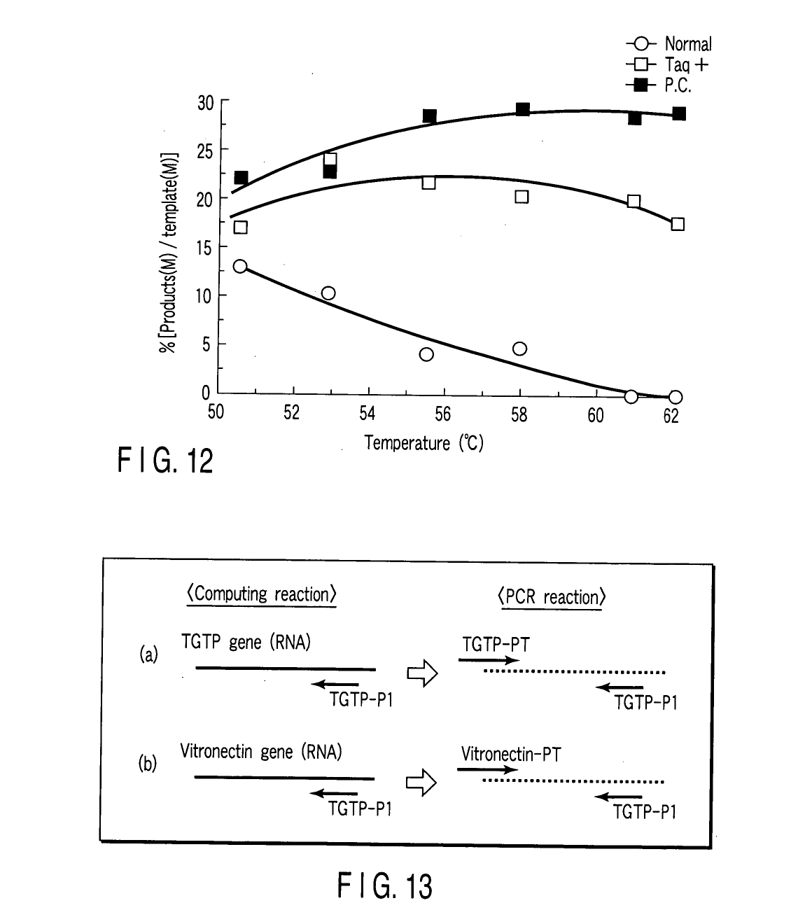

The assay performed for each reaction activity under the conditions using heat-resistant enzymes at form 50° C. to 62° C. showed that DNA dependent RNA polymerase activity becomes dramatically higher as higher temperature beyond 50° C. (FIG. 11), while RNA dependent DNA polymerase activity is almost stable at 50° C.-58° C. (FIG. 10), demonstrating the difficulty of setting the reaction temperature at 50° C. or higher. In addition, the experiments showed that the decrease of DNA dependent DNA polymerase activity according to higher temperature might be recovered by addition of Taq DNA polymerase (FIG. 12). AMV reverse transcriptase is known to have DNA polymerase activity against single stranded RNA or DNA template, as well as RNaseH activity to remove RNA strand from DNA-RNA hybrid (Baltimore et al. 1972, Champoux et al. 1984, Verma 1977), and, in addition, Taq polymerase is known to have exonuclease activity. It was experimentally demonstrated that the reaction would proceed without RNaseH if these enzymes used (data not shown).

Based on the considerations above, we decided that the computing reaction was performed using a reaction solution comprising AMV reverse transcriptase, TT7 RNA polymerase and Taq DNA polymerase as hardware under the condition maintained at constant temperature, 50° C.

(Assessment of the Specificity of Primer Elongation Reaction)

In the computing reaction, data input and operations are carried out by elongation reaction with primers using RNA and DNA as templates. Thus, it is very important to ensure the specificity of priming. Here, we carried out the experiment to assess the activity and specificity of primer elongation reaction in reverse transcription reaction with specifically designed primers using, as targets, in vitro-synthesized gene fragments for both TGTP/Mg21 gene (hereinafter called TGTP gene) and Vitronectin gene, which are known to be highly expressed in graft versus host disease (GVHR).

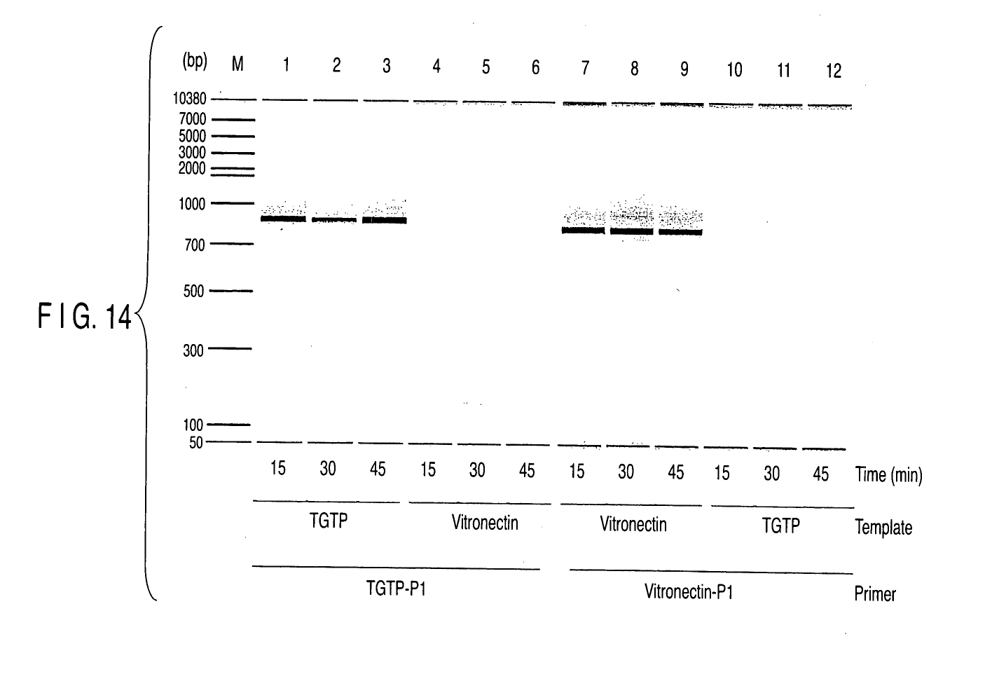

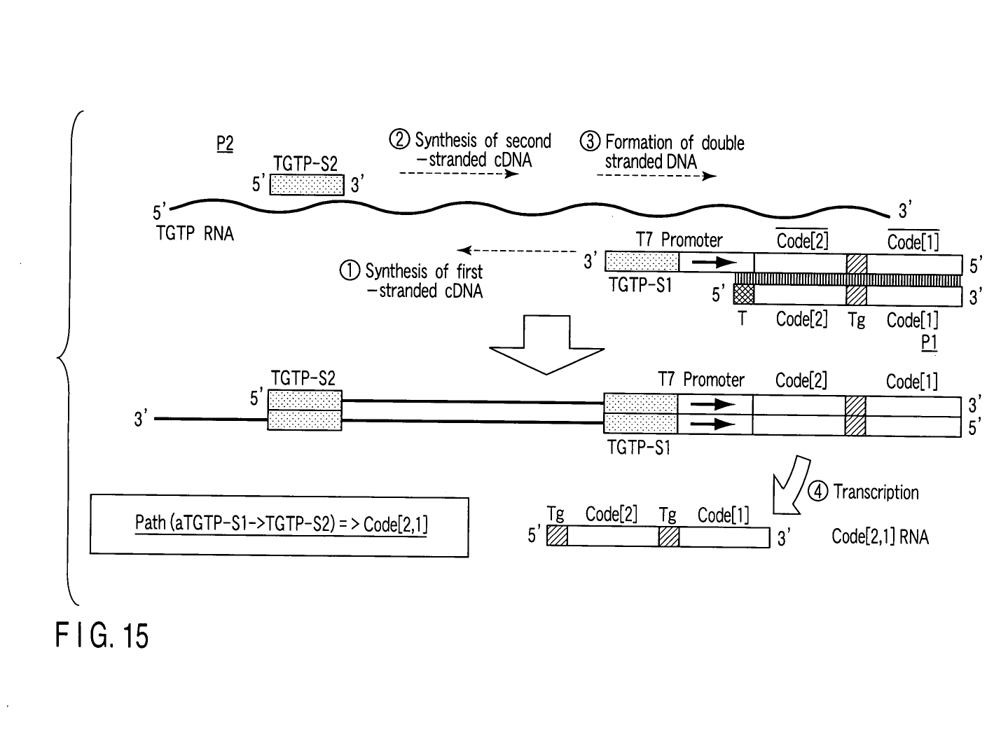

TGTP-P1 is a primer having TGTP-P1, which is specific sequence of TGTP gene, at 3′-end. To assess the elongation activity and specificity of this primer, the computing reaction was carried out with mixture of this primer and TGTP gene for 15, 30 and 45 min, and the resulting primer elongation product was applied to PCR amplification reaction (FIG. 13-(a)), followed by gel electrophoresis to detect the resulting amplified product, resulting in the band located at expected MW, 843 bp observed (FIG. 14, lane 1-3). When the similar experiment was performed using Vitronectin gene instead of TGTP gene, no bands were observed (FIG. 13-(b)). In the PCR amplification reaction, the elongated primer and the primer containing the 5′-terminal sequence of either TGTP or Vitronectin molecule, added as a template (TGTP-PT or Vitronectin-PT), were used as the primer pair (A). It may also cause the detection of purified cDNA elongated by mispriming. These results confirmed that primer TGTP-P1 specifically binds to the target region in TGTP gene and initiates the elongation reaction at least in the presence of TGTP gene and Vitronectin gene.

In the similar experiment using Vitronectin-P1, which is specific primer for Vitronectin gene RNA, a peak was observed at expected MW, 792 bp, only in the presence of vitronectin gene (lane 7˜12). This result confirms that this primer also provides specific priming only with the target region. However, smear signal observed suggests that the non-specific reactions also occur slightly.

Above results ensured the availability of TGTP-P1 and Vitronectin-P1 primers as specific primers in the hardware. Furthermore, the similar experiment performed under the condition at 37° C. resulted in primer-dimer-like bands and non-specific smear detected strongly, demonstrating again that the reaction condition at 50° C., developed here, is appropriate for the computing reaction (data not shown).

(Execution of Encoding Functions)

In the presence of specific RNA, an encoding function generates the corresponding coded RNA. First, it would be important to execute the encoding function to achieve the gene expression analysis program. Here, we designed the encoding functions for TGTP gene and Vitronectin gene RNA, and performed the experiment using them.

The structure of TGTP encoding function is showed in FIGS. 2-5A. In this function based on the underlying function A (See FIG. 6A.), aTGTP-S1 (complementary strand sequence to TGTP-S1) and TGTP-S2 containing in TGTP gene are used as arguments, and Code[2,1] sequence is used as a return value (Path (aTGTP-S1→TGTP-S2)=>Code[2,1]). In turn, the primer (P1), involved in the first strand cDNA synthesis, contains T7 promoter sequence and a coding sequence as well as sequence TGTP-S1, and the primer(P2), involved in the second strand cDNA synthesis, comprises sequence TGTP-S2. The transcription is expected to proceed as follows: in the presence of TGTP gene RNA, a reverse transcription reaction is led by P1, followed by the synthesis reaction of the second strand with S2, providing formation of double-stranded T7 promoter, and, resulting in code[2,1] sequence (aligned Code[2] and Code[1] sequences across the Tg sequence) RNA transcripted. In addition, to output coding RNA would be always added Tg sequence (5′-GGGAGA-3′) at its 5′-end because the transcription initiate site of T7 transcriptase is within the promoter sequence. Furthermore, it may be effective that to 5′-terminal sequence of P1 (complementary strand moiety of a coding sequence and a part of promoter sequence) is made hybridized with the complimentary oligo DNA to form double strand DNA because, if 5′-terminal sequence of P1 was still single stranded, unreacted P1 might bind to the output coding RNA and form hybrid, causing degrading of P1 by RNaseH activity, and, further more, when still single stranded P1 used for the reaction, no output RNA was actually detected in the reaction (data not shown).

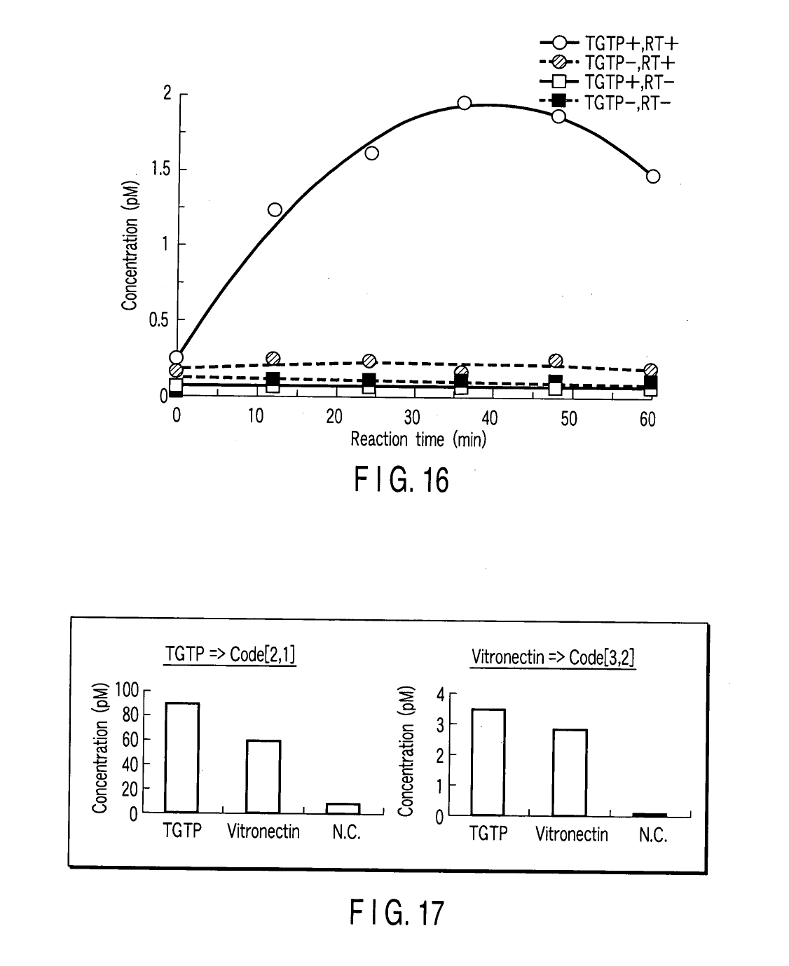

TGTP gene encoding function illustrated here was executed using hybrid of TGTP-T21 and aT21 oligo DNA as P1, and TGTP-S2 as P2 in the computing reaction solution to perform the quantitative experiment for RNA of output coding sequence, Code[2,1] (FIG. 16). The coding sequence RNA was detected by reverse transcription reaction and real-time PCR reaction for DNase I-treated computing reaction product. When TGTP was provided (open circle), increased coding sequence RNA was observed, while any change was not observed over the course of this experiment in the absence of TGTP (circle with diagonal line). Since insufficient treatment with DNaseI for the detection reaction might cause the coding sequence in P1 detected, the experiment was carried out also without addition of the enzymes in the reverse transcription reaction (open square or filled square), resulting in coding sequence almost undetected, suggesting that background was sufficiently low. Therefore, TGTP gene coding function was confirmed to be active in the computing reaction solution. The peak of synthesized coding sequence RNA was observed at around 40 min of reaction time and thereafter the amount decreased gradually, which may be attributed to the fact that RNA synthesis reaction activity decreases over time and RNA degrading reaction would exceed it. In this reaction, the concentration of input TGTP gene RNA was 0.17 nM, and the concentration of a coding sequence RNA product in the computing reaction solution was calculated based on the result of this quantification, resulting in 1.58 nM of 36 min-reaction-product. Multiple equivalent experiments (data not shown) showed that, in the case of 30 to 60 min reaction time, the amount of obtained output of coding sequence RNA was several-fold to dozen-fold more than in the reaction with TGTP gene.

The reaction specificity of encoding functions was assessed experimentally. The computing reaction was performed for 30 min with addition of TGTP gene RNA and Vitronectin gene RNA for encoding functions, or the same amount of water (N.C.) for negative control, and the concentration of the resulting coded sequence was measured (FIG. 17C). These computing reactions are expected to provide an output coding sequence only in the presence of TGTP gene sample given, while, actually, the signal of the coding sequence was observed also when Vitronectin gene provided. Similar experiments using Vitronectin gene encoding function also did not demonstrate any specificity (FIG. 17D).

(Reverse Transcription Reaction and Operation Reaction with the Path Across Multiple RNA Molecules)

When performing theoretical operation and gene expression analysis program with a neural network on the molecular computer, it is required to give the reaction to reverse transcript a multiple RNA molecules-comprising path. The reverse transcription for the multiple RNA molecules-comprising path is the process involving reverse transcription initiated by priming of primers to the first RNA molecule and further priming of 3′-end of the resulting cDNA to the second RNA molecule, in which RNaseH activity is important to remove the first RNA molecule.

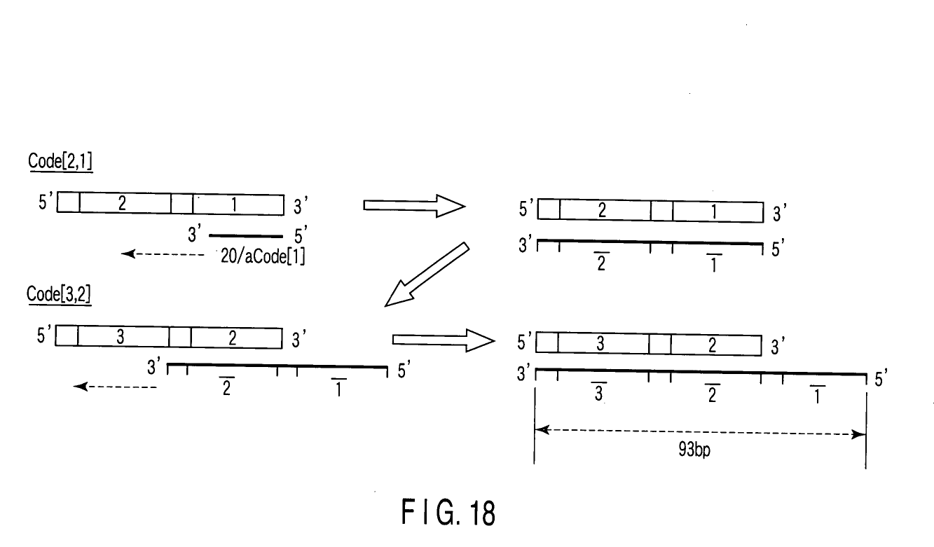

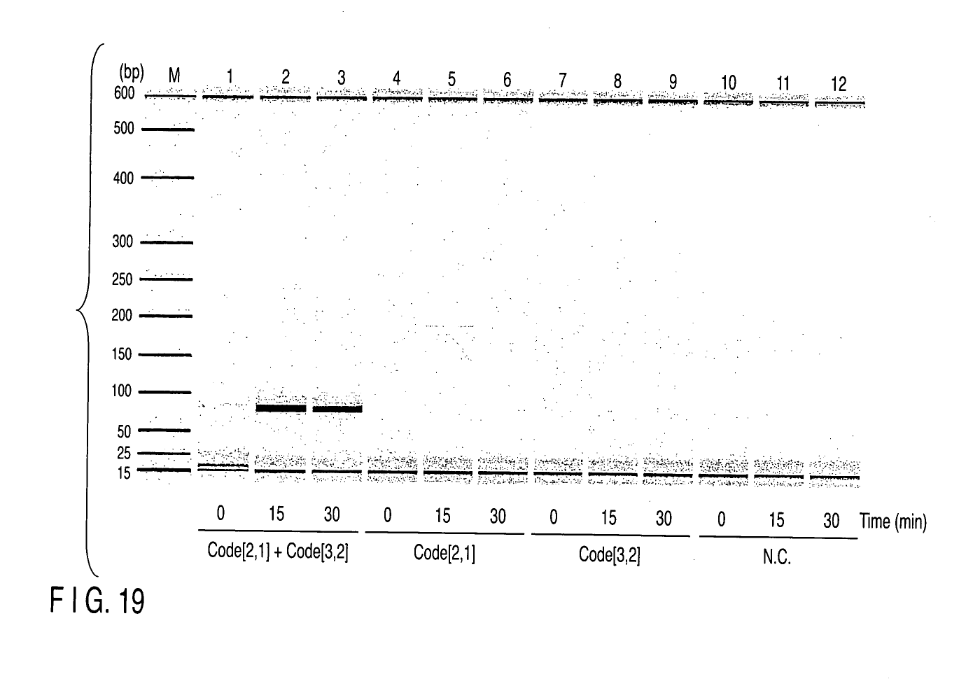

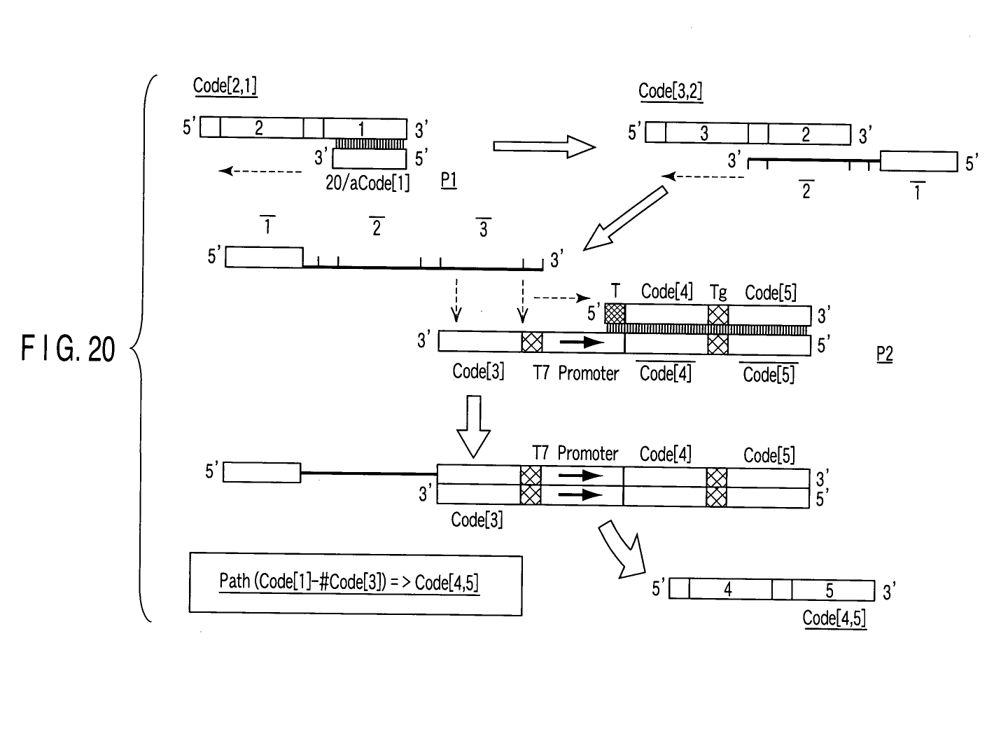

The experiment was performed to assess the reaction to reverse transcript the path in RNA across two RNA molecules, Code[1]→Code[2]→Code[3], using Code[2,1] and Code[3,2] RNA molecules, synthesized in vitro as described FIGS. 18, and 20/aCode[1] primer complementary to Code[1] sequence. To the computing reaction solution was added 20/aCode[1] primer and RNA sample and reacted for 0, 15 and 30 min. The resulting cDNA product was PCR-amplified and detected by electrophoresis, which demonstrated that the expected cDNA was formed when Code[2,1] and Code[3,2] RNA molecules were used and reacted for 15 min or more (FIG. 19).

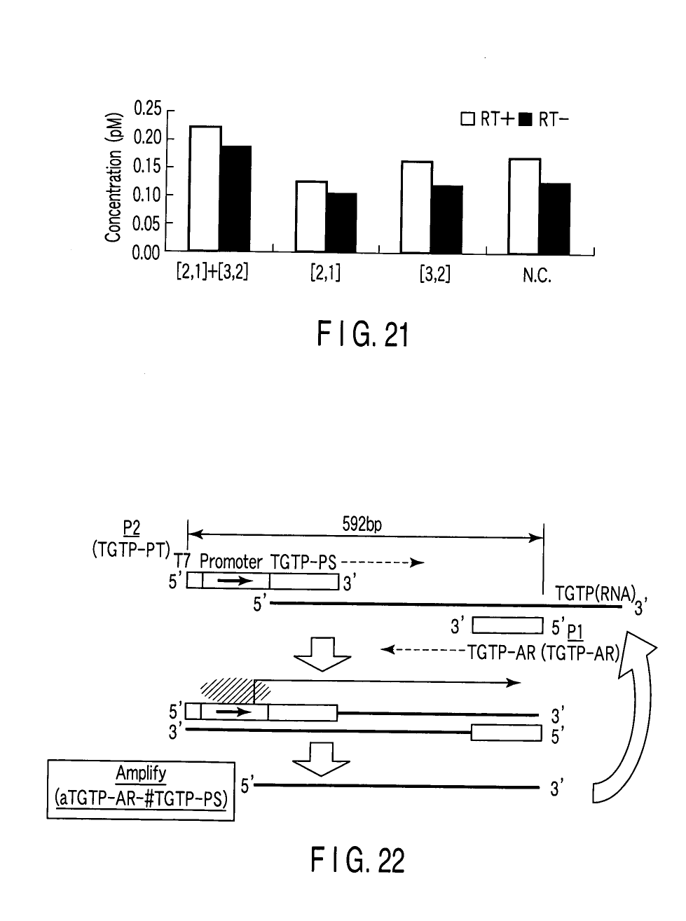

The feasibility of the reverse transcription reaction along with multiple RNA molecules demonstrated, the function using the path as an argument, “Path (Code[1]-# Code[3]),=>Code[4,5]”, was constructed based on the underlying function B (FIG. 20). When this function was combined with the function returning Code[2,1] using TGTP gene as an argument and the function returning Code[3,2] using Vitronectin gene as an argument, it was expected to achieve the theoretical operation program, “TGTP [symbol] Vitronectin [symbol] code[4,5]”, which returns Code[4,5] only when both TGTP gene and Vitronectin gene co-existing (See FIG. 7). Then, an experiment was performed using RNA samples used in the experiment of FIG. 19 to assess the reaction returning Code[4,5] RNA molecules resulted from formation of the path starting at Code[1] and ending at Code[3] only when Code[2,1] and Code[3,2] RNA molecules co-existing. For functions, 20/aCode[1] was used for P1 as same as the experiment of FIG. 19, and a hybrid formed with aC3-T45 and aT45 was used for P2. As a result, significant expression of Code[4,5] was not observed even if both Code[2,1] and Code[3,2] RNA molecules provided (FIG. 21). The expression of Code[4,5] was observed in the another experiment with direct addition of the complementary strand sequence oligo DNA of Code[3] instead of a coding sequence RNA molecule and 20/aCode[1] primer(data not shown), which suggests the inadequacy of the reaction efficiency and specificity in former experiment.

(Execution of Sense Strand RNA Amplifying Function)

Based on underlying function C: Amplify (a−# b [--add5 P] [--add3 Q]), the amplifying function for TGTP gene sequence was designed and the reaction was examined experimentally. TGTP-PT is the primer used in vitro synthesis of TGTP gene RNA, thus sequence TGTP-PS, which is located at 3′-end of this primer, is identical to 5′-end of TGTP gene RNA and further has T7 promoter sequence at its 5′-end. TGTP-AR primer comprises reverse complementary sequence to the 26 base-length region, starting at the position of 538^{th} base of in vitro synthesized TGTP gene RNA.

Combination of TGTP-PT and TGTP-AR primers provides the gene amplifying function “Amplify(a TGTP-AR-# TGTP-PS”, using TGTP gene as the argument, wherein pass of TGTP gene RNA was expected to lead to amplification of the sense strand RNA sequence, sandwiched between sequence TGTP-PS and TGTP-AR (FIG. 22).

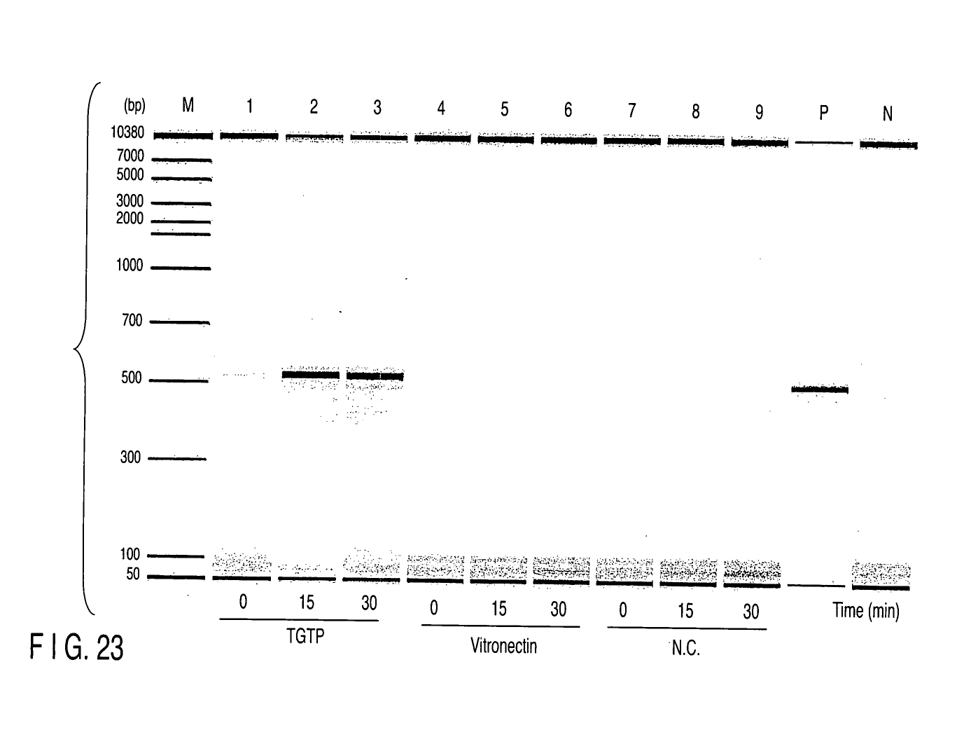

Using this TGTP gene sense strand RNA amplifying function, the computing reaction was performed for 0, 15 and 30 min with addition of either of in-vitro synthesized TGTP gene or Vitronectin gene, or addition of the same volume of water (N.C.) to detect the RNA products. The detection was performed as follows: the computing reaction product, which was treated with DNaseI to remove primer DNAs and intermediate DNAs, was applied to reverse transcription using TGTP-AR primer, followed by PCR-amplification using both TGTP-AR and TGTP-PT, and detected by gel electrophoresis (FIG. 23, lane M: marker). This method could also cause to detect the RNA molecules synthesized in non-specific reactions with TGTP-AR and TGTP-PT. The band of double stranded DNA having 592 base length was expected to be detected in the presence of the amplificated product of TGTP gene RNA through this RT-PCR, and actually its amount was increased over the reaction time, which demonstrated that the targeted RNA molecule in the reaction A was amplified (lane 1˜3). However, in comparing to positive control (lane P), smear signal was detected below the 592 bp band, suggesting the possibility of non-specific reactions occurring slightly. On the contrary, when Vitronectin gene RNA (FIG. 23, lane 4-6) or equal amount of water only (N.C.; FIG. 23, lane 7˜9) added, any signals were not detected. Smear signal located at less than 100 bp may be primer dimmer generated in the PCR reaction because such signals were also observed when equal volume of water added (lane N), instead of the sample.

Discussion

We carried out the experiments to implement the molecular computer simulating the retrovirus genome amplification reaction in a reaction system in vitro. In the hardware for this molecular computer, it was required to allow DNA dependent DNA polymerase, RNA dependent DNA polymerase, DNA dependent RNA polymerase and RNaseH activities to co-exist, in addition, it was essential to ensure the sufficient specificity for each reaction to execute the computing reaction correctly. In this study, the reaction condition fitted to above requirements was newly developed and applied to the experiments as hardware.

Executing the gene encoding reaction with this hardware confirmed the generation of the coding sequence RNA at the constant temperature for as short reaction time as 30˜40 min. This might be applied to gene expression measurement effectively as an easy-to-use gene encoding technique, as well as available as the input for the program of the higher leveled molecular computer, such as gene expression analysis. It is quite unlikely that there are any problems in the target specificity of the priming because gene specific sequence region of the first primers (P1), which were used in the encoding functions for TGTP gene and Vitronectin gene respectively, have already been confirmed the specificity for the primer elongation in FIG. 2-4. Also in the Amplify function, there was no problem about target specificity. Some effects from formation of primer dimmer and non-specific reaction with gene RNA may possibly contribute to insufficient specificity of the gene encoding reaction. When the promoter site was double stranded, the encoding function used here would output a coding sequence RNA identical to one output when the target gene was recognized, thus the promoter sequence in P1 double stranded by any non-specific reaction, wrong RNA, which cannot be distinguished from the appropriate output, may be transcripted. Calculation of the structure and stability of hybrid generated by both encoding functions and gene RNAs showed the possibilities, for P1s of both encoding functions, that the promoter P2 and gene RNA stably hybridize to the promoter sequence in P1 with the promoter sequence of P1, and gene RNA fractions act as non-specific primers (data not shown). The sequence of primers used in the encoding functions should be designed carefully, because these primers contain long single stranded DNA sequence comprising the target-specific sequence and T7 promoter sequence, providing more chances to cause non-specific hybridization such as primer dimer, which would make the promoter sequence double stranded, resulting in inappropriate output of coding sequence RNA. In addition, the functions used in the retro viral type molecular computer reported herein are defined with added oligo DNAs, thus addition of multiple primers to single reaction solution would enable to execute multiple functions simultaneously. While this may realize more complicated programs such as gene expression analysis, however, if allowing functions to co-existing in single reaction solution, it would be required to add more kind of oligo DNAs to the reaction solution, resulting in occurrence of more serious problems involving non-specific reactions. Accordingly, it is desired to develop the technique to design the appropriate nucleic acid sequence when constructing advanced programs. For example, the orthonormalized sequences described above are preferred.

This study showed that the reaction required to implement the retro viral type molecular computer may be achieved in vitro. Furthermore, TGTP gene and Vitronectin gene, which were targeted in the study, are counted to be applied as marker genes to do gene diagnosis for graft versus host reaction (GVHR) after transplant surgeries. In this study, gene expression analysis program was designed to consist of the encoding functions using these genes as arguments and the functions receiving the output and then executing the operation functions, a part of which was showed experimentally to be evidently executable. The system of this molecular computer is expected to provide the establishment of technology to allow each molecule within a test tube to analyze the expression patterns of multiple genes autonomously and output the results with only operations to execute the reactions in single tube at the constant temperature, which may be expected to be applied for the simple and accurate gene diagnosis technology. Furthermore, in the future, it is also expected to develop into the study to execute the similar molecular computer system in living cells, thus the findings from the studies may indicate the new direction of molecular computer studies.

(Multilayering of Multiple Functions)

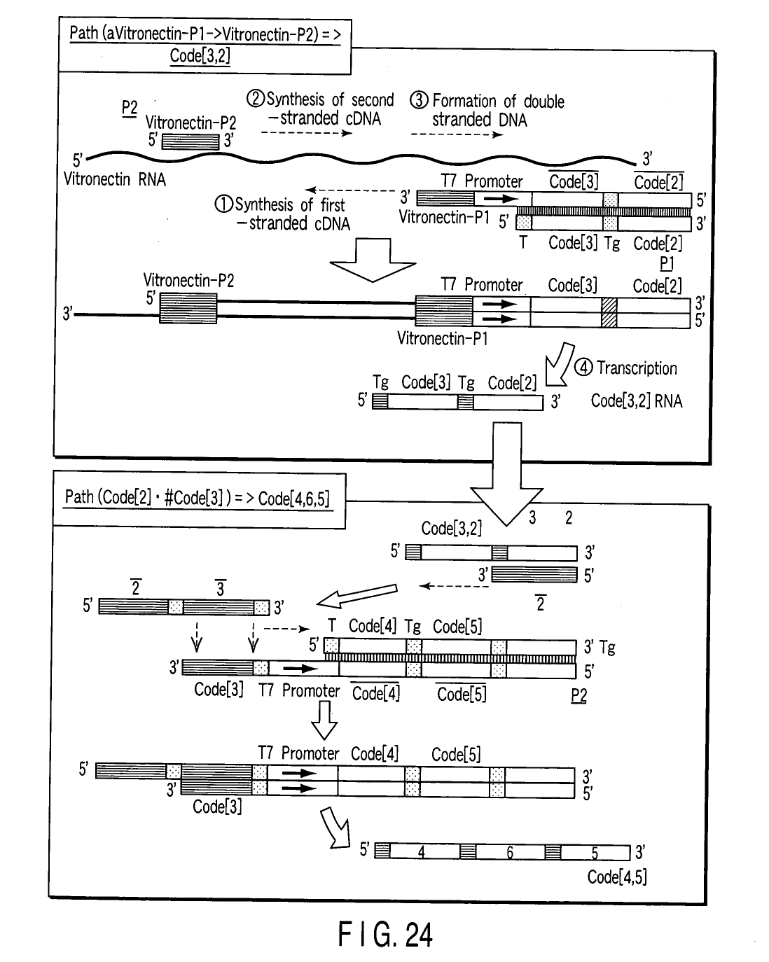

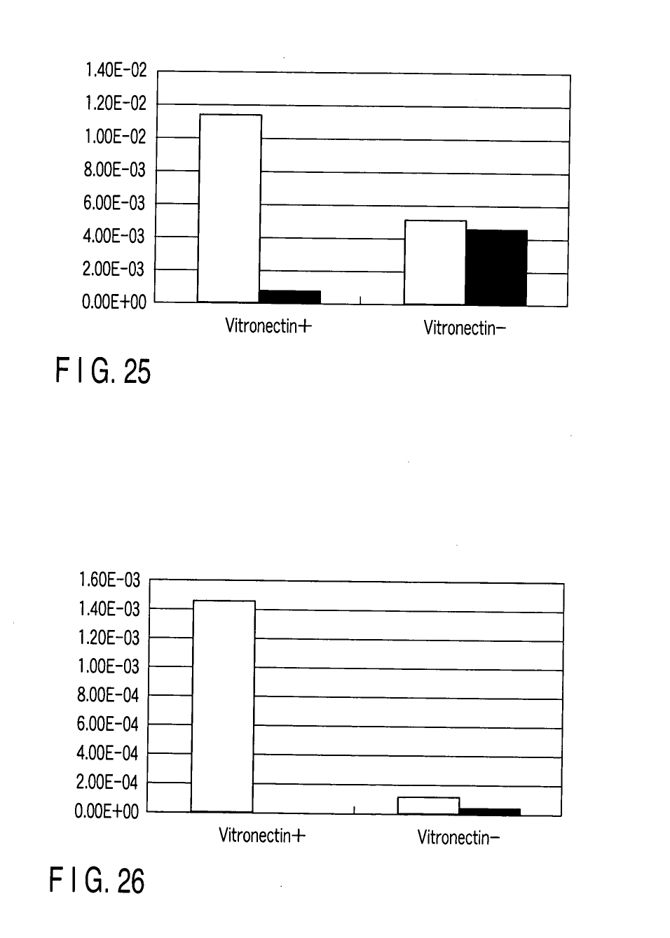

In the molecular computer, a return value of one function may be used as an argument for another function. An experiment was conducted to determine the semantics of a program comprising an encoding function outputting Code[3,2] RNA sequence in the presence of Vitronectin gene and another function outputting Code[4,6,5] RNA sequence in the presence of a return value from the former functions in single computing reaction solution. The reaction is summarized in FIG. 24. In the experiment, a computer reaction was carried out with addition of either Vitronectin gene as an input for this program (Vitronectin +) or only equal volume of water without Vitronectin gene (Vitronectin −), and then detection was carried out for the resulting RNA. The detection was performed by real-time PCR method for Code[4,6,5] sequence using primer pair corresponding to Code[4] and Code[5]. The result is showed in FIG. 25. In the experiment, detected RNA products were prepared with (RT+; clear bar) or without (RT−; filled bar) adding the enzyme in normal concentration at the stage of reverse transcription separately, and as a result, when Vitronectin RNA added as an input, the amount of output in a RT+ sample was much larger than a RT-sample (Vitronectin +), while, when adding no input, there was not clear difference between RT+ and RT− (Vitronectin −). The resulting output from a RT− sample was background of the experiment, thus the difference of detected results between RT+ and RT− may reflect the amount of RNA molecule obtained as an output. Furthermore, similar detection reactions for Code[3,2] RNA, which is an intermediate product, using the above products demonstrated that the concentration of Code[3,2] also increased when Vitronectin added (FIG. 26, clear bar:RNA+, filled bar:RNA−).

The above results demonstrated that the program comprising 2 types of functions illustrated FIG. 24 is executed evidently, and a combination of multiple functions may be executed in single computing reaction solution. More complicated programs may be achieved by functions multilayered, thus it is important to use a return value from one function as an argument of another function practically.

What is claimed is:

1. An information processing method carrying out operations with functions receiving an argument and returning a return value through chemical reactions of molecules, comprising:
(a) inputting a first encoded nucleic acid defined in correspondence to a first degradable single stranded nucleic acid as an argument:
(b) carrying out an operation with functions defined in correspondence to chemical reactions of operator nucleic acids based on said argument:
(c) obtaining a second encoded nucleic acid defined in correspondence to a second single stranded nucleic acid as a return value.

2. An information processing method according to claim 1, wherein said second single stranded nucleic acid is a degradable nucleic acid.

3. An information processing method according to claim 2, comprising carrying out an operation with a further function using said second encoded nucleic acid obtained from the (c) as a first encoded nucleic acid and obtaining a further second encoded nucleic acid as a return value.

4. An information processing method according to claim 2, comprising obtaining a second encoded nucleic acid as a return value through execution of multiple operations with said functions.

5. An information processing method which extracts a calculation result of a program described with combination of said functions, the arguments and the return values by carrying out an operation with multiple functions following said program using an information processing method according to claim 4.

6. An information processing method according to claim 1, comprising that:
inputting in said (a) is to add said first single stranded nucleic acid to a reaction solution containing said operator nucleic acid and suitable enzymes:
operation in said (b) is to induce a chemical reaction among said operator nucleic acid, said suitable enzyme and said first single stranded nucleic acid:
a return value in said (c) is obtained as a reaction product of said chemical reaction.

7. An information processing method according to claim 6, wherein
said first single stranded nucleic acid and said second first single stranded nucleic acid are RNA,
said chemical reactions are a synthesis reaction, an amplification reaction, a reverse transcription reaction, a transcription reaction and a degrading reaction, each of which is a synthesis reaction or an amplification reaction with an enzyme having DNA dependent DNA polymerase activity, a reverse transcription reaction with an enzyme having RNA dependent DNA polymerase activity and a transcription reaction with an enzyme having DNA dependent RNA polymerase activity, and a degrading reaction with RNaseH respectively.

8. An information processing method according to claim 7, wherein said operator nucleic acid has one or more sequences selected from sequences working as a primer for said first single stranded nucleic acid, promoter sequences and sequences acting as a primer for any nucleic acid.

9. An information processing method according to claim 8, wherein said function is a function returning RNA of a specified sequence x as a return value when RNA containing a path starting, at sequence a through sequence b input as an argument;
said operator nucleic acids are two nucleic acids, a first operator nucleic acid having a promoter sequence in 5′-end direction, a reverse complementary sequence to x at the downstream and a complementary sequence to a at the 3′-terminal, and a second operator nucleic acid, having sequence b;
said chemical reaction is a reaction involving reverse transcription of said RNA starting at complementary sequence to a in said first operator nucleic acid, providing reverse transcripted single stranded DNA, which is bound by sequence b in said second operator nucleic acid, leading to a synthesis reaction of a second strand DNA starting at 3′-end of said second operator nucleic acid, followed by hybridization between said second strand DNA and a promoter sequence in said first operator nucleic acid, providing a transcription reaction of the sequence x located downstream of said promoter sequence, resulting in RNA molecules of the sequence x synthesized as a reaction product.

10. An information processing method according to claim 8, wherein said function is a function returning RNA of a specified sequence x as a return value when RNA containing a path starting at sequence a and ending at b input as an argument;
said operator nucleic acids are two nucleic acids, a first operator nucleic acid having sequence a, and a second operator nucleic acid having a promoter sequence in 5′-end direction, a sequence x downstream of the promoter sequence and sequence b at the 3′-termianl;
said chemical reaction is a reaction involving reverse transcription of said RNA starting at sequence a in said first operator nucleic acid, providing reverse transcripted single stranded DNA, which is bound by sequence b in said second operator nucleic acid, leading to a further synthesis reaction starting at 3′-end of said single stranded DNA, followed by hybridization of the promoter sequence in said second operator nucleic acid, providing a transcription reaction of the sequence x located downstream of said promoter sequence, resulting in RNA molecules of the sequence x synthesized as a reaction product.

11. An information processing method according to claim 8, wherein, when RNA starting at sequence a trough b input as an argument, said function returns said RNA or RNA containing said RNA and any additional sequence as a return value;
said operator nucleic acids are two nucleic acids, a first operator nucleic acid having a complementary sequence to a and a optional complementary sequence to sequence q, and a second operator nucleic acid having a promoter sequence in 3′-end direction, an optional sequence p at the 3′-termianl and sequence b at the 3′-termianl;
said chemical reaction is a reaction involving reverse transcription of said RNA starting at complementary sequence to a in said first operator nucleic acid, providing reverse transcripted single stranded DNA, which is bound by sequence b in said second operator nucleic acid, leading to a further synthesis reaction starting at 3′-end of said single stranded DNA, followed by hybridization of the promoter sequence in said second operator nucleic acid, providing a transcription reaction of sequence located downstream of said promoter sequence, resulting in said RNA molecules or the RNA containing said RNA and additional sequence p or q synthesized as a reaction product.

12. An information processing method according to claim 8, wherein, when RNA starting at sequence a trough b input as an argument, said function returns RNA having a reverse complementary sequence to said RNA or RNA containing the complementary sequence to said RNA and any additional sequence as a return value;
said operator nucleic acids are two nucleic acids, a first operator nucleic acid having a promoter sequence in 3′-end direction, an optional sequence p at the 3′-end and a complementary sequence to a at the 3′-end, and a second operator nucleic acid having sequence b, an optional sequence q at the 3′-end;
said chemical reaction is a reaction involving reverse transcription of said RNA starting at the complementary sequence to a in said first operator nucleic acid, providing reverse transcripted single stranded DNA, which is bound by sequence b in said second operator nucleic acid, leading to a synthesis reaction starting at 3′-end of said second operator nucleic acid, followed by hybridization between said DNA and the promoter sequence in said first operator nucleic acid, providing a transcription reaction of a sequence x located downstream of said promoter sequence, resulting in RNA molecules having a reverse complementary sequence to said RNA or RNA molecules having a reverse complementary sequence to RNA containing said RNA and additional sequence p or q synthesized as a reaction product.

13. An information processing method according to claim 8, wherein said function is a function always returning RNA of sequence x as a return value without requiring RNA as an argument;
said operator nucleic acids are a first operator nucleic acid having a promoter sequence in 3′-end direction and sequence x downstream of the promoter, and the second operator nucleic acid having a promoter sequence in the 5′-end direction and a complementary sequence to x downstream of the promoter.
said chemical reaction is a reaction wherein said first operator nucleic acid binds to said second operator nucleic acid, leading to transcription reaction of sequence x located downstream of the promoter sequence, resulting in RNA molecules of sequence x synthesized as a reaction product.

14. An information processing method extracting a calculation result of said program by carrying out an operation with multiple functions following a method according to claim 8.

15. A kit for carrying out information processing using nucleic acid molecules, containing operator nucleic acids for performing an operation with desired functions.

16. A kit according to claim 15, wherein said operator nucleic acid is a nucleic acid having one or more sequence selected from sequences acting as a primer for said first single stranded nucleic acid, promoter sequences and sequences acting as a primer for any nucleic acid.

17. A kit according to claim 15, wherein said kit additionally contains a suitable reaction solution and suitable enzymes.

18. A kit according to claim 17, wherein
said suitable reaction solution is a buffer suitable for a synthesis reaction, an amplification reaction, a reverse transcription reaction, a transcription reaction and a degrading reaction, and
said suitable enzymes are an enzyme having DNA dependent DNA polymerase activity, an enzyme having RNA dependent DNA polymerase activity and an enzyme having DNA dependent RNA polymerase activity, and RNaseH.

19. A molecular computer for carrying out an operation using an information processing method according to claim 1, consisting of a container comprising operator nucleic acids for carrying out an operation with desired functions, a suitable reaction solution and suitable enzymes.

20. A molecular computer according to claim 19, wherein said first operator nucleic acid is a nucleic acid having one or more sequence selected from sequences acting as a primer for said first single stranded nucleic acid, promoter sequences and sequences acting as a primer for any nucleic acid, said suitable reaction solution is a buffer suitable for a synthesis reaction, an amplification reaction, a reverse transcription reaction, a transcription reaction and a degrading reaction, said suitable enzymes are an enzyme having DNA dependent DNA polymerase activity, an enzyme having RNA dependent DNA polymerase activity and an enzyme having DNA dependent RNA polymerase activity and RNaseH.


SEQUENCE LISTING (29 sequences)

SEQ ID NO: 1 - DNA, 20 bp, Artificial Sequence
  TGTP-specific oligonucleotide primer
cagatatatatggtcccacc

SEQ ID NO: 2 - DNA, 20 bp, Artificial Sequence
  TGTP-specific oligonucleotide primer
acttactatcgcatggctta

SEQ ID NO: 3 - DNA, 24 bp, Artificial Sequence
  TGTP-specific oligonucleotide primer
caggatttgaacatgtctgtggat

SEQ ID NO: 4 - DNA, 26 bp, Artificial Sequence
  TGTP-specific oligonucleotide primer
gcttgtcttctaaggactcatcattg

SEQ ID NO: 5 - DNA, 20 bp, Artificial Sequence
  TGTP-specific oligonucleotide primer
ggggatgaatttctactttg

SEQ ID NO: 6 - DNA, 20 bp, Artificial Sequence
  TGTP-specific oligonucleotide primer
agagtgaacactgattggaa

SEQ ID NO: 7 - DNA, 20 bp, Artificial Sequence
  Vitronectin-specific oligonucleotide primer
tttgtctccagagaagaaat

SEQ ID NO: 8 - DNA, 20 bp, Artificial Sequence
  Vitronectin-specific oligonucleotide primer
gctaggaacctacaacaact

SEQ ID NO: 9 - DNA, 20 bp, Artificial Sequence
  Vitronectin-specific oligonucleotide primer
gtaccccaaacttatccaag

SEQ ID NO: 10 - DNA, 20 bp, Artificial Sequence
  Vitronectin-specific oligonucleotide primer
gtagggaggattcacagagt

SEQ ID NO: 11 - DNA, 25 bp, Artificial Sequence
  oligonucleotide coding sequence
tgaagtcaccacaacacacagtaca

SEQ ID NO: 12 - DNA, 25 bp, Artificial Sequence
  oligonucleotide coding sequence
gacaaacaccccgaatacaaacagc

SEQ ID NO: 13 - DNA, 25 bp, Artificial Sequence
  oligonucleotide coding sequence
agtatcgaagcgtgtgtctgaagat

SEQ ID NO: 14 - DNA, 25 bp, Artificial Sequence
  oligonucleotide coding sequence
caaaagagttaggatgggagctgga

SEQ ID NO: 15 - DNA, 25 bp, Artificial Sequence
  oligonucleotide coding sequence
tcgatatgggtggtacatgagaggt

SEQ ID NO: 16 - DNA, 25 bp, Artificial Sequence
  oligonucleotide coding sequence
ctccgctcctctcttcattccctag

SEQ ID NO: 17 - DNA, 93 bp, Artificial Sequence
  nucleic acid primer
ctgaggttatcttggtctggggagatctccctatagtgagtcgtattactgaggttatcttggtctggggagacagatatatatggtcccacc

SEQ ID NO: 18 - DNA, 99 bp, Artificial Sequence
  nucleic acid primer
tgtactgtgtgttgtggtgacttcatctcccgctgtttgtattcggggtgtttgtctctccctatagtgagtcgtattacagatatatatggtcccacc

SEQ ID NO: 19 - DNA, 99 bp, Artificial Sequence
  nucleic acid primer
gctgtttgtattcggggtgtttgtctctcccatcttcagacacacgcttcgatacttctccctatagtgagtcgtattatttgtctccagagaagaaat

SEQ ID NO: 20 - DNA, 65 bp, Artificial Sequence
  nucleic acid primer
atagggagagacaaacaccccgaatacaaacagcgggagatgaagtcaccacaacacacagtaca

SEQ ID NO: 21 - DNA, 65 bp, Artificial Sequence
  nucleic acid primer
atagggagaagtatcgaagcgtgtgtctgaagatgggagagacaaacaccccgaatacaaacagc

SEQ ID NO: 22 - DNA, 49 bp, Artificial Sequence
  nucleic acid primer
gatgcataatacgactcactatagggagaggggatgaatttctactttg

SEQ ID NO: 23 - DNA, 49 bp, Artificial Sequence
  nucleic acid primer
gatgcataatacgactcactatagggagagtaccccaaacttatccaag

SEQ ID NO: 24 - DNA, 20 bp, Artificial Sequence
  nucleic acid primer
tgtactgtgtgttgtggtga

SEQ ID NO: 25 - DNA, 110 bp, Artificial Sequence
  nucleic acid primer
acctctcatgtaccacccatatcgatctccctccagctcccatcctaactcttttgtctccctatagtgagtcgtattagggagaagtatcgaagcgtgtgtctgaagat

SEQ ID NO: 26 - DNA, 65 bp, Artificial Sequence
  nucleic acid primer
atagggagacaaaagagttaggatgggagctggagggagatcgatatgggtggtacatgagaggt

SEQ ID NO: 27 - DNA, 141 bp, Artificial Sequence
  nucleic acid primer
acctctcatgtaccacccatatcgatctcccctagggaatgaatagaggagcggagtctccctccagctcccatcctaactcttttgtctccctatagtgagtcgtattagggagaagtatcgaagcgtgtgtctgaagat

SEQ ID NO: 28 - DNA, 96 bp, Artificial Sequence
  nucleic acid primer
atagggagacaaaagagttaggatgggagctggagggagactccgctcctctattcattccctaggggagatcgatatgggtggtacatgagaggt

SEQ ID NO: 29 - DNA, 6 bp, Artificial Sequence
  oligonucleotide Tg sequence
gggaga